(19) United States
(12) Patent Application Publication
(10) Pub. No.: US 2026/0249079 A1
(43) Pub. Date: Aug. 27, 2026

(54) WEARABLE DEVICE FOR SLEEP APNEA TREATMENT SYSTEM, AND ASSOCIATED METHODS

(71) Applicant: Restera, Inc., Santa Clara, CA (US)

(72) Inventors: William Welch, Sunnyvale, CA (US); David Herron, Los Angeles, CA (US); Richard W O'Connor, Atherton, CA (US)

(21) Appl. No.: 19/647,764

(22) Filed: Apr. 14, 2026

Related U.S. Application Data
(63) Continuation of application No. PCT/US24/52050, filed on Oct. 18, 2024.
(60) Provisional application No. 63/592,059, filed on Oct. 20, 2023.
(60) Provisional application No. 63/622,903, filed on Jan. 19, 2024.

(51) Int. Cl. A61N 1/36; A61N 1/378

(52) U.S. Cl. CPC A61N 1/3611; A61N 1/3787

(57) ABSTRACT

The present technology is generally directed to wearable devices for wearable devices for sleep apnea treatment systems. The wearable devices of the present technology can be configured to be worn comfortably and securely around at least a portion of a wearer's neck and/or one or more other portions of the wearer's anatomy. At least some of the wearable devices described herein include one or more power transmission devices configured to transmit power to one or more implantable devices positioned within a patient. Additionally, or alternatively, the wearable devices of the present technology can include one or more sensors configured to obtain data associated with a breathing obstruction experienced by a wearer.

### CROSS-REFERENCE TO RELATED APPLICATIONS

The present application is a continuation of International Patent Application No. PCT/US 24/52050, filed Oct. 18, 2024, titled “WEARABLE DEVICE FOR SLEEP APNEA TREATMENT SYSTEM, AND ASSOCIATED METHODS,” which claims priority to U.S. Provisional App. No. 63/592,059 filed Oct. 20, 2023, titled “WEARABLE DEVICE FOR SLEEP APNEA TREATMENT SYSTEM, AND ASSOCIATED METHODS” and to U.S. Provisional App. No. 63/622,903, filed Jan. 19, 2024, titled “WEARABLE DEVICE FOR SLEEP APNEA TREATMENT SYSTEM, AND ASSOCIATED METHODS”, the entireties of which are hereby incorporated by reference.

### TECHNICAL FIELD

The present technology is directed to wearable devices for sleep apnea treatment systems, and associated methods.

### BACKGROUND

Obstructive sleep apnea (OSA) is a medical condition in which a patient's upper airway is occluded (partially or fully) during sleep, causing sleep arousal. Repeated occlusions of the upper airway may cause sleep fragmentation, which in turn may result in sleep deprivation, daytime tiredness, and/or malaise. More serious instances of OSA may increase the patient's risk for stroke, cardiac arrhythmias, high blood pressure, and/or other disorders.

OSA may be characterized by the tendency for soft tissues of the upper airway to collapse during sleep, thereby occluding the upper airway. OSA is typically caused by the collapse of the patient's soft palate, oropharynx, tongue, epiglottis, or combination thereof, into the upper airway, which in turn may obstruct normal breathing and/or cause arousal from sleep.

Some treatments have been available for OSA including, for example, surgery, constant positive airway pressure (CPAP) machines, and electrically modulating muscles or related nerves associated with the upper airway to move the tongue (or other upper airway tissue). Surgical techniques have included procedures to remove portions of a patient's tongue and/or soft palate, and other procedures that seek to prevent the tongue from collapsing into the back of the pharynx. These surgical techniques are very invasive. CPAP machines seek to maintain upper airway patency by applying positive air pressure at the patient's nose and mouth. However, these machines are uncomfortable, cumbersome, and may have low compliance rates.

Some proposed OSA treatments include implanting one or more devices within a patient to provide electrical modulation that at least partially addresses the OSA. Many such implantable devices include an implanted power source, and implantation frequently requires invasive surgical intervention. Other implantable devices are powered from outside the body; however, existing techniques for transmitting power to patients are often bulky and/or uncomfortable, leading to low patient compliance. For example, external power transmission devices that are adhered to a patient's skin with an adhesive can be uncomfortable (e.g., such as irritating the skin and/or pulling on the skin). This is particularly the case for individuals with facial hair. It can also be difficult to repeatably and/or consistently align external power transmission devices with one or more implantable devices.

### BRIEF DESCRIPTION OF THE DRAWINGS

FIG. 1A is a side sectional view depicting a patient's upper airway.

FIG. 1B is a partially schematic illustration of representative neural structures and musculature of the patient's lower jaw and neck.

FIG. 1C is a perspective view of motion planes of the patient's lower jaw and neck.

FIG. 2A is a block diagram illustrating elements of a system for treating sleep disorders in accordance with embodiments of the present technology.

FIG. 2B is a side view the wearable device of FIG. 2A configured in accordance with embodiments of the present technology.

FIG. 2C is a side view the wearable device of FIG. 2A configured in accordance with embodiments of the present technology.

FIG. 3A is a perspective view of a wearable device configured in accordance with embodiments of the present technology.

FIGS. 3B-3F are perspective views of the wearable device of FIG. 3A showing representative movement of the wearable device with the wearer's neck.

FIGS. 3G and 3H illustrate a representative wearable device configured in accordance with further embodiments of the present technology.

FIGS. 4A and 4B are perspective views of another wearable device configured in accordance with embodiments of the present technology.

FIGS. 5A and 5B are perspective views of another wearable device configured in accordance with embodiments of the present technology.

FIGS. 6-11 are perspective views of respective wearable devices configured in accordance with embodiments of the present technology.

FIGS. 12A and 12B are front and rear perspective views, respectively, of another wearable device configured in accordance with embodiments of the present technology.

FIGS. 13A and 13B are perspective views of another wearable device configured in accordance with embodiments of the present technology.

FIG. 13C is a plan view of select features of the wearable device of FIGS. 13A and 13B, with other features of the wearable device omitted for the purpose of clarity.

FIGS. 14A-14D are partially schematic perspective views of a wearable device positioned around the wearer's neck, in accordance with embodiments of the present technology.

FIG. 15 is a top view of another wearable device configured in accordance with embodiments of the present technology.

FIGS. 16A and 16B are perspective views of another wearable device configured in accordance with embodiments of the present technology.

FIG. 16C is a top view of a connector portion and support members of the wearable device of FIGS. 16A and 16B with other aspects of the wearable device omitted for illustrative clarity.

FIG. 16D is a perspective view of an electronics housing and support members of the wearable device of FIGS. 16A and 16B with a cover portion of the electronics housing and other aspects of the wearable device omitted for illustrative clarity.

FIG. 16E is a top cross-sectional view of the wearer's neck with the wearable device of FIGS. 16A and 16B positioned therearound in accordance with embodiments of the present technology.

### DETAILED DESCRIPTION

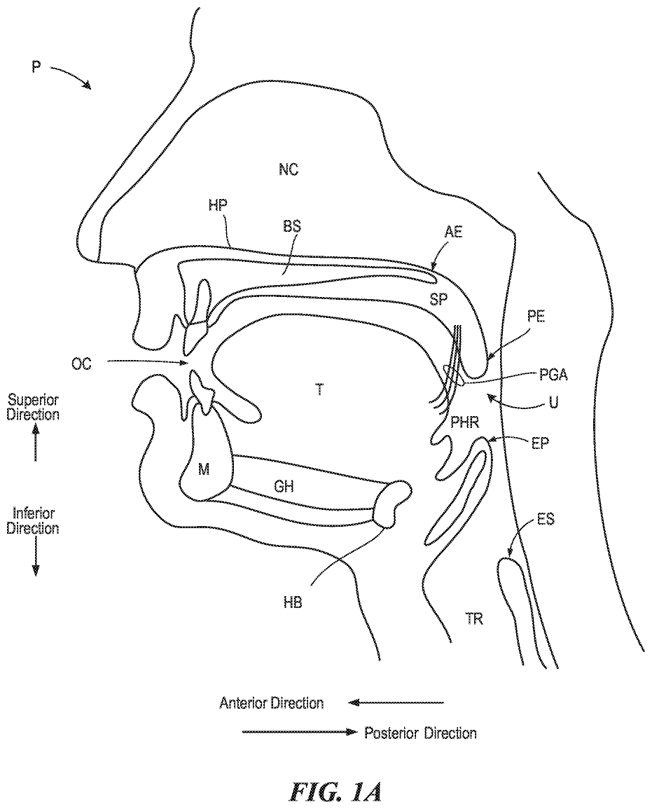

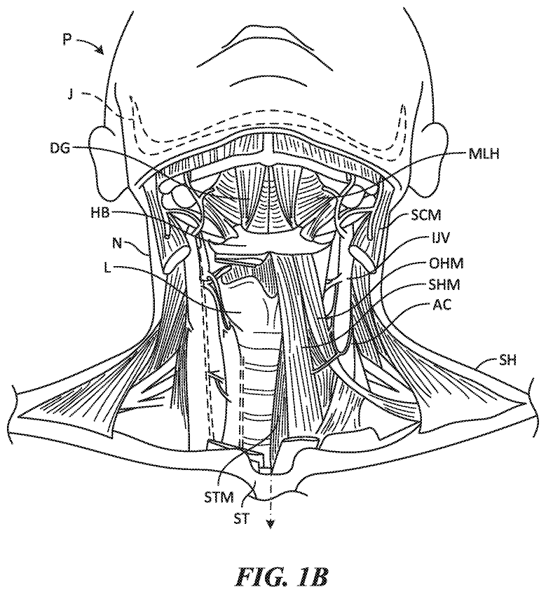

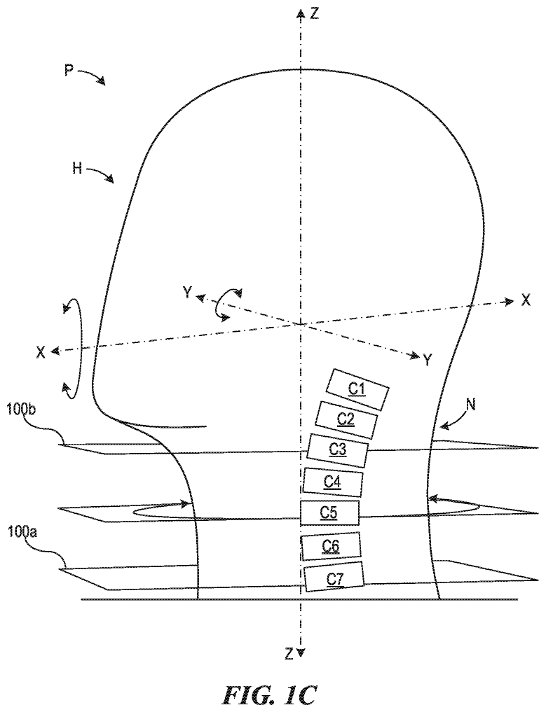

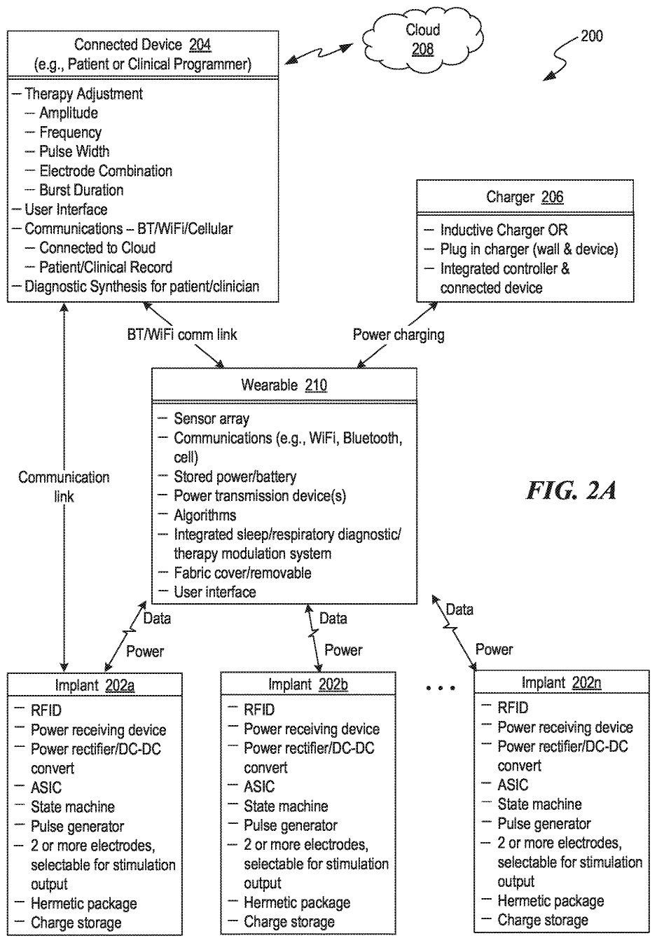

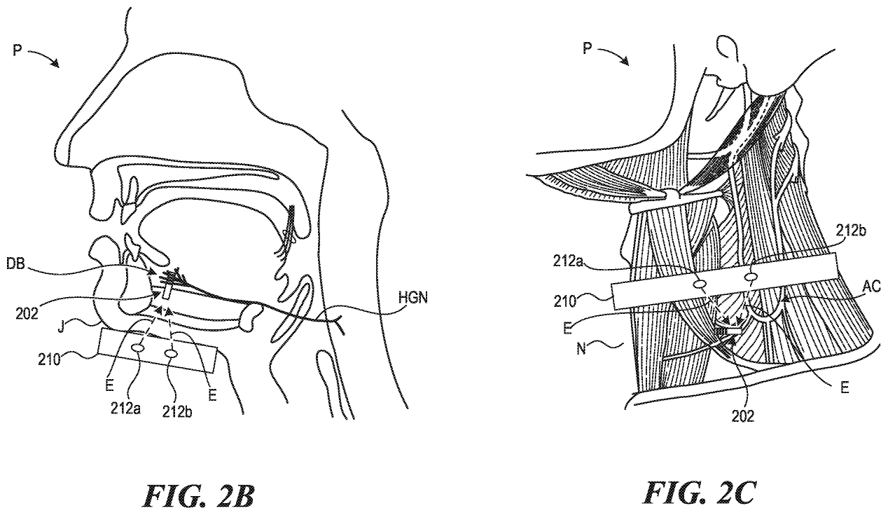

The present technology is discussed under the following headings for ease of readability:
Heading 1:“Introduction”
Heading 2:“Overall Patient Physiology” (with a focus on FIGS. 1A-1C)
Heading 3:“Representative Wearable Devices and Associated Sleep Apnea Treatment System Elements” (with a focus on FIGS. 2A-16E)
Heading 4:“Examples”
Heading 5:“Closing Remarks”

Although embodiments of the present technology are described under the selected headings indicated above, other embodiments of the technology can include elements discussed under multiple headings. Accordingly, an embodiment discussed under a particular heading is not necessarily limited to only the elements discussed under that heading.

### 1. Introduction

Electrical modulation therapy for obstructive sleep apnea (OSA) typically includes delivering a modulation signal (e.g., an electrical signal, one or more pulses, etc.) that modulates nerves and/or muscles to cause (i) the tongue and/or other soft tissue to move. and/or (ii) change the tissue tone (e.g., tighten or stiffen the tissue without muscular contraction or extension that induces movement). The electrical modulation can accordingly remove an obstruction of the upper airway, and/or prevent the tongue or other soft tissue from collapsing or obstructing the airway. As used herein, the terms “modulate” and “stimulate” are used interchangeably to mean having an effect on a nerve, a muscle, and/or other tissue that in turn has an effect on one or more motor functions (e.g., a breathing-related motor function).

Representative methods and apparatuses for reducing the occurrence and/or severity of a breathing disorder, such as OSA, OSA with complete concentric collapse (“CCC”), central sleep apnea, and/or the like, are disclosed herein. In some embodiments, a signal delivery device is implanted at least proximate to or in contact with one or more target tissues of the patient's upper airway, such as one or more nerves that innervate a muscle in the patient's airway and/or oral cavity. The signal delivery device can be implanted in the patient via a minimally invasive percutaneous injection. The signal delivery device can receive power wirelessly from an external or “wearable” device and use that power to generate and/or deliver accurately targeted modulation signals (e.g., electrical signals, stimulation pulses, etc.) to the target tissues, thereby improving the patient's upper airway patency and/or improve the tone of the tissue of the intraoral cavity to treat sleep apnea. The external device can include one or more mouthpiece portions, collar portions, chinstrap portions, pillow portions, mattress overlay portions, and/or one or more other suitable wearable structures described herein.

Representative target tissues include nerves such as the ansa cervicalis nerve and/or the hypoglossal nerve, which are located adjacent and/or around the oral cavity or in the neck. Modulating the ansa cervicalis nerve can induce caudal traction (e.g., of the trachea), lower or depress the hyoid bone, and/or stabilize or stiffen the tongue and/or soft tissues of the upper airway. This, in turn, can reduce or prevent tissue collapse and/or other airflow obstructions in the patient's airway, thereby improving airflow through the upper airway and mitigating or even alleviating the breathing obstruction. For example, because the tongue is attached to the hyoid bone, lowering the hyoid bone can (i) draw the tongue downwardly/inferiorly and prevent, or at least partially prevent, the tongue and/or associated tissues from obstructing the patient's airway, and/or (ii) improve airflow through the upper airway. Modulating the hypoglossal nerve can cause the patient's tongue to move anteriorly/forward and/or improve tissue tone to prevent the tongue and/or other soft tissues in the airway from collapsing onto the back of the patient's pharynx and/or into the upper airway. Such movement of potentially obstructive tissue in the upper airway/pharynx is expected to improve the patient's sleep by mitigating or alleviating the obstruction. Further target tissues can include one or more muscles innervated by the hypoglossal nerve or the ansa cervicalis nerve (e.g., one or more of the patient's infrahyoid strap muscles, including the sternohyoid muscles and/or the sternothyroid muscles), the glossopharyngeal nerve, the pharyngeal branches of the glossopharyngeal nerve, the pharyngeal plexus, the C2 or C3 spinal nerve, a lateral part of the epidural space at the C1, C2, and C3 vertebral bodies, the pharyngeal branches of the glossopharyngeal nerve, and/or other suitable and/or therapeutically effective targets. Accordingly, the devices and associated methods disclosed herein can improve the patient's sleep by moving and/or stabilizing potentially obstructing tissue in the upper airway/pharynx. More specifically, applying the modulation signal to one or more portions of the ansa cervicalis nerve and/or directly to one or more of the patient's infrahyoid strap muscles can (i) cause the patient's hyoid bone to move inferiorly (e.g., caudal traction), (ii) increase a stiffness of the patient's pharyngeal wall, and/or (iii) otherwise at least partially or fully prevent soft tissue collapse that would otherwise have an obstructive effect on the patient's upper airway.

Many embodiments of the technology described below may take the form of computer-or machine-or controller-executable instructions, including routines executed by a programmable computer or controller. Those skilled in the relevant art will appreciate that the technology can be practiced on computer/controller systems other than those shown and described below. The technology can be embodied in a special-purpose computer, controller or data processor that is specifically programmed, configured or constructed to perform one or more of the computer-executable instructions described below. Accordingly, the terms “computer” and “controller” as generally used herein refer to any suitable data processor and can include Internet appliances and hand-held devices (including palm-top computers, wearable computers, tablets, cellular or mobile phones, multi-processor systems, processor-based or programmable consumer electronics, network computers, minicomputers and the like). Information handled by these computers can be presented at any suitable display medium, including a liquid crystal display (LCD). In some embodiments. manufacturers or other suitable entities can provide instructions to practitioners for executing the methods disclosed herein. Manufacturers can also program devices of the disclosed systems to carry out at least some of these methods.

The present technology can also be practiced in distributed environments, where tasks or modules are performed by remote processing devices that are linked through a communications network. In a distributed computing environment, program modules or subroutines may be located in local and remote memory storage devices. Aspects of the technology described below may be stored or distributed on any suitable computer-readable media, including one or more ASICs, (e.g., with addressable memory), as well as distributed electronically over networks. Data structures and transmissions of data particular to aspects of the technology are also encompassed within the scope of the embodiments of the technology.

### 2. Overall Patient Physiology

Representative embodiments described herein include wearable and/or other external devices configured to provide power to one or more implantable signal delivery devices having electrodes that can be positioned to deliver one or more modulation signals to one or more specific target locations, e.g., specific nerves and/or specific positions along a nerve. Such locations include locations along the patient's ansa cervicalis nerve, hypoglossal nerve, and/or vagus nerve, as well as those nerves that innervate muscles of the airway (e.g., palatal, oropharyngeal, laryngeal, omohyoid, sternohyoid, sternothyroid, thyrohyoid, nasal, lingual, pharyngeal, infrahyoid, diaphragmatic, and/or intercostal muscles). The target location can be identified with respect to any of, or any combination of, intrinsic or extrinsic muscles, associated nerve branches and/or portions thereof, and/or other physiological features. For example, some target locations can be within the patient's neck, such as at least proximate to the ansa cervicalis nerve, omohyoid muscle, sternohyoid muscle, sternothyroid muscle, and/or thyrohyoid muscle. Other target locations can be located superior to the neck and/or within or at least proximate to the patient's oral cavity, such as at least proximate to the hypoglossal nerve, at least proximate to the genioglossus muscle, and/or within the genioglossus muscle.

FIG. 1A is a side sectional view depicting an upper airway of a patient P. The patient P has a hard palate HP which overlies the tongue T and forms the roof of the oral cavity OC (e.g., the mouth). The hard palate HP includes bone support BS, and thus does not typically deform during breathing. The soft palate SP, which is made of soft tissue such as membranes, fibrous material, fatty tissue, and muscle tissue, extends rearward (e.g., in a posterior direction) from the hard palate HP toward the back of the pharynx PHR. More specifically, an anterior end AE of the soft palate SP is anchored to a posterior end of the hard palate HP, and a posterior end PE of the soft palate SP is unattached. Because the soft palate SP does not contain bone or hard cartilage, the soft palate SP is flexible and may collapse onto the back of the pharynx PHR and/or flap back and forth (e.g., especially during sleep).

The pharynx PHR, which passes air from the oral cavity OC and the nasal cavity NC into the trachea TR, is the part of the throat situated inferior to (below) the nasal cavity NC, posterior to (behind) the oral cavity OC, and superior to (above) the esophagus ES. The pharynx PHR is separated from the oral cavity OC by the palatoglossal arch PGA, which runs downward on either side to the base of the tongue T. Although not labeled for simplicity, the pharynx PHR includes the nasopharynx, the velopharynx, the oropharynx, and the laryngopharynx. The nasopharynx lies between the base of the cranium and the soft palate SP. The velopharynx is the section of the nasopharynx bounded ventrally by the soft palate. The oropharynx lies behind the oral cavity OC and extends from the soft palate SP to the pharyngoepiglottic fold. The oropharynx opens anteriorly into the oral cavity OC. The anterior portion of the oropharynx includes the base of the tongue T. A flap of connective tissue called the epiglottis EP closes over the glottis (not labeled for simplicity) when food is swallowed, to prevent aspiration. The laryngopharynx is the portion of the pharynx that divides anteriorly into the larynx and posteriorly into the esophagus, and is bounded by the pharyngoepiglottic fold superiorly and the upper esophageal sphincter inferiorly. Below the tongue T is the lower jaw or mandible M, and the geniohyoid muscle GH, which is one of the muscles, in addition to the infrahyoid strap muscles, that controls the movement of the hyoid bone HB. Modulating one or more of the patient's infrahyoid strap muscles (and/or a nerve innervating one or more of the patient's infrahyoid strap muscles) can lower the hyoid bone HB, including in an anterior or posterior direction such as shown using dashed-line arrows in FIG. 1B, and produce a corresponding movement of at least the base of the patient's tongue T. Lowering the base of the patient's tongue T can open the patient's airway, and/or reduce or prevent tissue collapse that at least partially obstructs the patient's airway, to increase airflow through the oral cavity OC and address OSA and/or other breathing obstructions.

FIG. 1B is a partially schematic illustration of representative neural structures and musculature of the patient's jaw J and neck N. The omohyoid muscle OHM extends between the hyoid bone HB and the scapula. The sternohyoid muscle SHM extends between the hyoid bone HB and the sternum ST, and the sternothyroid muscle STM extends between the sternum ST and the patient's thyroid cartilage TH. The ansa cervicalis AC, and related branches emanating from the ansa cervicalis AC, enervate the omohyoid muscle OHM, the sternohyoid muscle SHM, and the sternothyroid muscle STM. The ansa cervicalis AC can also extend at least partially parallel to and/or around the patient's internal jugular vein IJV. FIG. 1B also illustrates the patient's mandible M, mylohyoid muscle MLH, and digastric muscle DG (more specifically, the anterior belly of the digastric muscle DG), as well as the sternothyroid muscle STM, sternocleidomastoid muscle SCM, and the sternohyoid muscle SHM. The sternohyoid muscles SHM and the sternothyroid muscles STM extend over (e.g., anterior to) the patient's larynx L. The muscles described above are contained within the patient's oral cavity OC, neck N, and/or shoulder SH. By positioning and activating minimally invasive electrodes at least proximate to one or more of the foregoing neural structures and/or associated musculature, embodiments of the present technology can direct one or more modulation signals to (e.g., single nerves or portions of nerves associated with specific muscles or movements) and/or the associated musculature to control, reduce, and/or eliminate the effects of OSA and/or other breathing obstructions.

FIG. 1C is a perspective view of a head H and the neck N of the patient P. The head H and/or neck N are highly mobile and can move (e.g., rotate) relative to roll X, pitch Y, and/or yaw Z axes. The movement of the head H and the neck N are interrelated, but movement along the neck N is not always uniform. For example, when turning to the left, an upper portion of the neck N proximate the head H (represented by second plane 100b) can rotate or flex more (e.g., rotate or flex through a greater angle) than a lower portion of the neck N (represented by first plane 100a). The neck N can rotate in both the first plane 100a and the second plane 100b but at least a portion of the rotation in the second plane 100b can also be relative and/or in addition to the rotation in the first plane 100 a. The first and second planes 100a, b are used for ease of reference. In practice, it will be appreciated that movement and/or rotation of individual cervical vertebra C1-C7 in the patient's neck N (shown schematically) can be interrelated and/or in response to movement and/or rotation of one or more others of the cervical vertebra C1-C7 about the roll X, pitch Y, and/or yaw Z axes. For example, rotating the C7 cervical vertebra about the yaw axis Z can cause at least a corresponding rotation of one or more of the C1-C6 cervical vertebra. As another example, rotating the head H in one direction (e.g., to the right) can cause each of the cervical vertebra C1-C7 can rotate in that direction (e.g., to the right), with C1 rotating by a greater amount than C2, C2 rotating by a greater amount than C3, etc.

Some wearable devices are configured to restrict or prevent movement of the neck N to consistently and repeatably position the wearable device relative to one or more devices implanted within or proximate to the neck N. However, devices that restrict neck movement are often uncomfortable and lead to low patient compliance. As described in greater detail below, wearable devices configured in accordance with embodiments of the present technology are expected to track movement of various portions of the patient's neck, including overall movement (e.g., rotation in the first plane 100a and the second plane 100b) and relative movement (e.g., rotation in the second plane 100b relative to at least some or all rotation in the first plane 100a). This is expected to improve patient comfort, which is expected to enhance compliance, and it is expected to maintain an at least approximately consistent and/or repeatable position of the power source relative to one or more devices implanted within or proximate to the neck N to keeps a desired distance between these devices (e.g., reduces changes in the distance between these devices) and enhance power transmission efficiency and/or efficacy of the therapy.

### 3. Representative Wearable Devices and Associated Sleep Apnea Treatment System Elements

FIG. 2A is a block diagram illustrating elements of a system 200 for treating sleep disorders in accordance with embodiments of the present technology. The system 200 can include a wearable device 210, a charger 206, one or more implants or implantable devices (e.g., a first implantable device 202a, a second implantable device 202b ... an nth implantable device 202n; referred to collectively as “implantable devices 202”) and a connected device or programmer 204. In general, the programmer 204 can transmit instructions for generating a modulation signal (e.g., signal delivery or waveform parameters) to the wearable device 210, the wearable device 210 can transmit the instructions and power to the implantable device(s) 202, and individual ones of the implantable devices 202 can collectively and/or independently generate the modulation signal according to the transmitted instructions and apply the modulation signal to a patient via electrodes carried by the implantable device(s) 202. At least some elements of the system 200 (e.g., the wearable device 210, the charger 206, the implantable devices 202, etc.) can be at least generally similar or identical in structure and/or function to one or more elements described in U.S. Pat. App. No. Ser. No. 17/851,718, the entirety of which is hereby incorporated by reference.

In some embodiments, the implantable devices 202 can include one or more capacitors and/or other devices configured to store a charge (e.g., for no more than 1 second, 2 seconds, 3 seconds, 4 seconds, 5 seconds, 10 seconds, 20 seconds, 30 seconds, 1 minute, 2 minutes, 3 minutes, 4, minutes, or 5 minutes). In some embodiment, one or more of the implantable devices 202 do not include a battery, power capacitor (e.g., a super capacitor), and/or other power storage element configured to store a charge for extended periods of time (e.g., at least 1 hour, 6 hours, 12 hours, 1 day, 1 week, 1 month, etc.) or powering the implantable devices 202 for prolonged periods of time in the absence of wirelessly delivered power. In other embodiments, one or more of the implantable devices 202 include one or more power storage elements configured to storage a charge for extended periods of time (e.g., at least 1 hour, 6 hours, 12 hours, 1 day, 1 week, 1 month, etc.). Such implantable devices can be configured for use with wearable devices that omit power transmission devices. Individual implantable devices 202 can be implanted in a patient to deliver a modulation signal to one or more portions of the hypoglossal nerve HGN (including the anterior branches AB and/or the distal brachiated portions DB (FIG. 2B)), the genioglossus muscle GG, the ansa cervicalis muscle, and/or one or more other modulation targets described herein.

The programmer 204 can include a patient-operated programmer and/or a clinician-operated programmer and can be configured to control one or more characteristics of the modulation signal delivered to the patient. In a representative embodiment, the programmer 204 can include a therapy adjustment module configured to select one or more of the electrodes carried by the implantable device(s) 202 and adjust (e.g., increase or decrease) an amplitude, frequency, pulse width, and/or burst duration, adjust whether the electrode is active or inactive, and/or any other suitable signal delivery parameter. Additionally, the programmer 204 can synthesize information (e.g., diagnostic and/or feedback information) received from a user, the wearable device 210, and/or the individual implantable devices 202 and can adjust one or more of the signal delivery parameters based at least partially on the synthesized information. For example, the programmer 204 can be configured to direct the modulation signal to specific distal brachiated portions DB (FIG. 2B) based, at least in part, on a tissue collapse pattern of the patient.

The programmer 204 can transmit the signal delivery parameters to the implantable device(s) 202 directly and/or via the wearable device 210. For example, the programmer 204 can be connected to individual implantable devices 202 and/or the wearable device 210 via a wired or wireless communication link, such as WiFi, Bluetooth (“BT”), cellular connectivity, and/or any other suitable communication link. In these and other embodiments, the programmer 204 can be connected to the “cloud” 208 and/or other computer service(s), e.g., to upload data received from the wearable device's 210 sensors and/or to download information to the wearable device 210 and/or the implantable device(s) 202. In these and other embodiments, the programmer 204 can include a display and/or a user interface. A user (e.g., the patient, the clinician, and/or other suitable user) can interact with and/or otherwise control one or more aspects of the programmer 204 via the user interface, e.g., to manually adjust one or more of the signal delivery parameters, to read data received from the wearable device 210 sensors, provide one or more inputs corresponding to a tissue collapse pattern, and/or carry out other tasks.

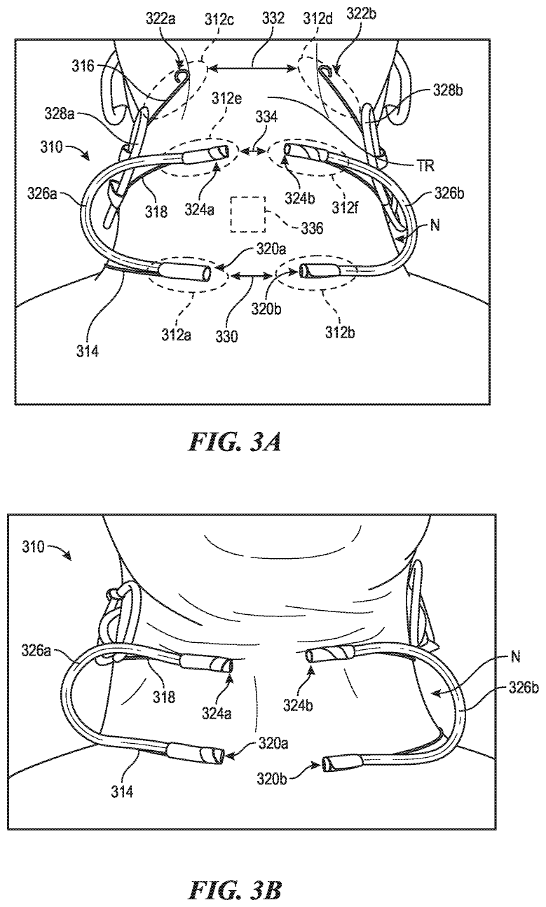

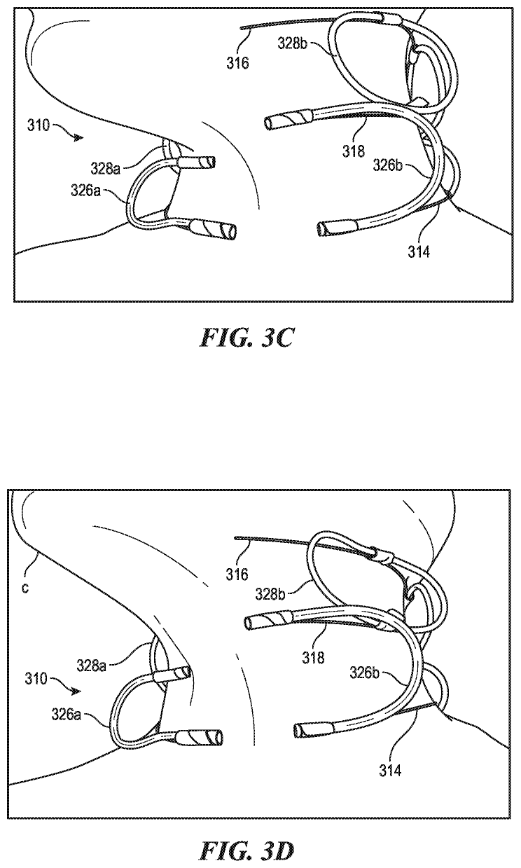

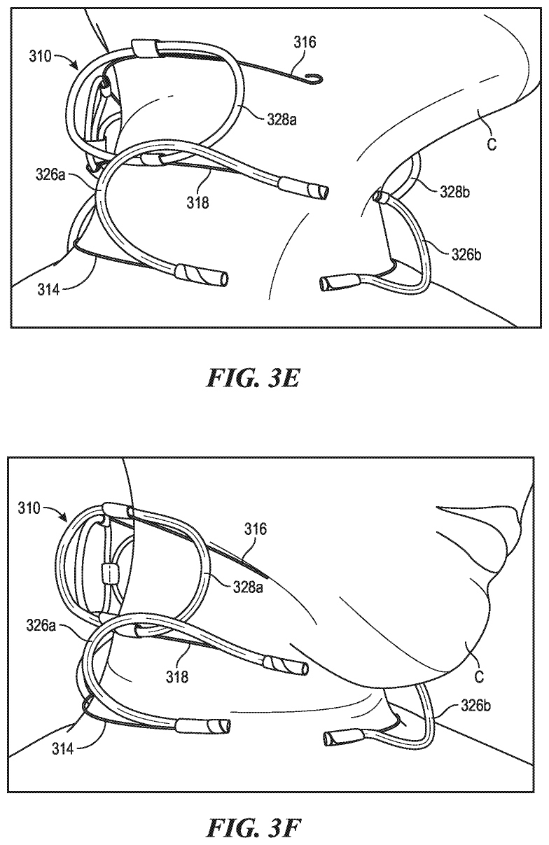

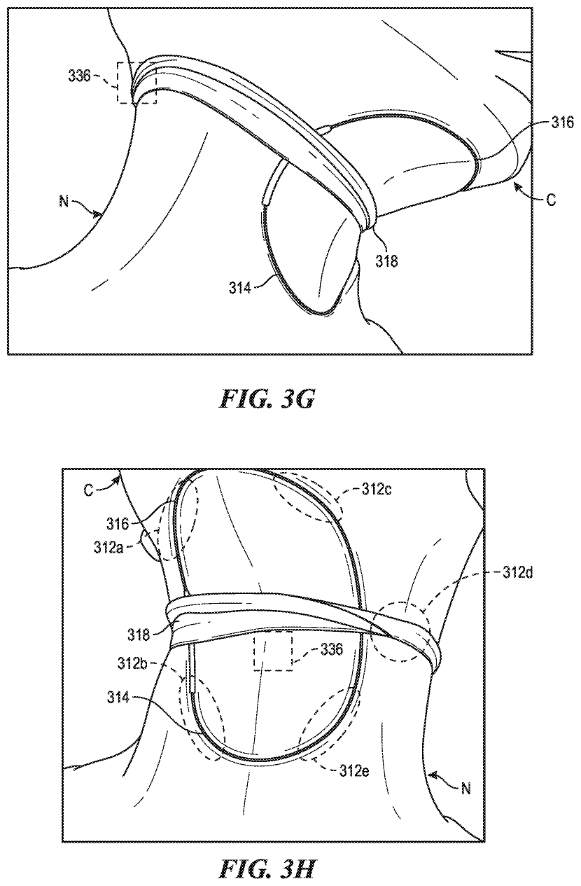

The wearable device 210 can have any of the features and/or form factors described herein with reference to FIGS. 3A-16E. The wearable device 210 can include one or more sensors (e.g., a single sensor, an array of sensors, and/or other suitable sensor arrangements) configured to collect data associated with a patient. Representative data received from the patient can include respiratory rate, sleep state, wake state, heart rate, audio signals (corresponding to audible snoring, tracheal sounds, hypopnea events, and/or apnea events), body temperature, head orientation/position, saturated blood oxygen levels, air flow levels, thyroid movement, trachea movement, tongue movement, and/or photoplethysmography (PPG) data, among others, each of which can be received by a corresponding type of sensor (e.g., heart rate data from a heart rate sensor, head orientation/position data from an accelerometer, etc.). These data can be received via one or more corresponding sensors (e.g., body temperature from a temperature sensor, audio signals from a microphone or other audio sensor, etc.), and can correspond to a measure of the patient's respiratory performance, sleep state, wake state, and/or other suitable metrics, for example, metrics that are used to rate the patient on the Apnea-Hypopnea Index (AHI). Additionally, or alternatively, the wearable device 210 can receive data from the individual implantable devices 202, e.g., using backscatter, pulse width modulation, frequency modulation, and/or one or more other suitable techniques. For example, the implantable devices 202 can transmit a receipt to indicate that power has been received, and what magnitude the power is. This information can be used to autoregulate (up or down) the output of the implantable device's pulse generator, e.g., the transmitted signal and phase.

The wearable device 210 can further include a power source (e.g., a stored power device such as battery), one or more power transmission devices configured to transmit power and/or signal delivery parameters to the implantable device(s) 202, and one or more algorithms configured to control one or more aspects of the operation of the wearable device 210. Individual sensors can collect data associated with the patient, such as a patient's sleep state and/or respiratory performance. The one or more algorithms can be configured to adjust at least one of the signal delivery parameters based at least partially on the data collected by the sensors. In a representative embodiment, the wearable device 210 can include an integrated sleep, respiratory diagnostics, and/or therapy modulation system configured to adjust or otherwise control one or more delivery parameters of the modulation signal delivered to the patient based on the collected sleep state and/or respiratory performance data, e.g., via one of more algorithms. In these and/or other embodiments, the algorithms can include a placement feedback system configured to provide feedback to the wearer if the wearable device 210 is not seated properly about the wearer's anatomy. For example, the placement feedback system can provide an indication of whether at least one of the power transmission devices is adequately or optimally aligned with the power receiving device associated with one of the implantable signal delivery devices. This can improve power transmission efficiency and patient comfort by reducing heat build-up associated with power transmission. As another example, the placement feedback system can provide an indication of whether the patient is wearing the wearable device 210 and/or whether the wearable device 210 is properly seated against one or more portions of the patient's anatomy. This can help to allow the wearable device 210 to follow movement of the patient's head and/or neck. As yet another example, the placement feedback system can determine whether the wearable device 210 is longitudinally, rotationally, and/or angularly aligned with one or more portions of the patient's anatomy and, if not, provide an indication that the patient should reposition and/or reorient the wearable device 210. This can help position the power transmission device proximate to the implanted signal delivery devices and enhance power transmission efficiency.

In some embodiments, the wearable device 210 can further include a cover or housing, at least a portion of which may be removeable to, e.g., expose an interior or interior portion of the wearable device 210. In these and other embodiments, cover can include fabric, or any other suitable material. Optionally, the wearable device 210 can include a reduced-scope and/or simplified user interface configured to allow a user to interact with and/or otherwise control one or more of the elements of the wearable device 210, e.g., without using the programmer 204. For example the wearable device user interface may allow the user to check a charging status of the power source, power on and/or off the wearable device 210, adjust one or more of the signal delivery parameters, configured and/or verify therapy delivery, select one or more therapy presets, confirm and/or verify placement of the wearable device, etc. The user interface can include one or more input and/or devices, such as one or more buttons, dials, switches, display screens, touch screens, touch sensors, speakers, lights, haptic feedback devices, etc., that the user can interact with and/or that are configured to provide feedback and/or other information to a user regarding the operation of the wearable device 210. In at least some embodiments, for example, the user interface can operate with the placement feedback system to provide feedback to the wearer if the wearable device 210 is not seated properly about the wearer's anatomy.

The charger 206 for the wearable device 210 can be configured to supply power to the wearable device's 210 power source. The charger 206 can include a wireless (e.g., inductive) charger, a wired charger (e.g., wall-plug, charging cable, etc.), and/or any other suitable charger or charging device. Optionally, the charger 206 can include an integrated controller and/or a connected device, e.g., to control the charging of the wearable device 210 and/or to upload/download data to the wearable device 210 while the wearable device 210 is charging.

The one or more implantable devices 202 can each include an RFID component (e.g., a unique RFID tag that can be used to identify and/or locate the associated implantable device 202a-n), a power receiving device (e.g., one or more RF power antennas, one or more inductive coils, etc.), a power rectifier/DC-DC converter, circuitry (e.g., one or more application-specific integrated circuits (ASICs), a state machine, etc.), a signal generator, and two or more electrodes that are each individually selectable to deliver a modulation signal to a patient. The power receiving device can receive power from the power transmission component (e.g., one or more RF power antennas, one or more inductive coils, etc.) of the wearable device. The power rectifier/DC-DC converter can be operably coupled to the electrode receiver antenna and can be configured to transmit the received power to the signal generator. Additionally, each of the implantable devices 202 can receive, via the power receiving device and/or one or more other communication components, information regarding one or more of the delivery parameters of the modulation signal to be generated by the signal generator and/or delivered to the patient via at least one of the electrodes of the implantable device(s) 202. The circuitry can include machine-readable instructions associated with the operation of the implantable device(s) 202. For example, the circuitry can include instructions that, when executed, can cause the signal generator to generate the modulation signal having the signal delivery parameter(s) received via the electrode receiver antenna. In these and other embodiments, the power receiving device and/or the one or more other communication components can be used to transmit information associated with the implantable device 202 to the wearable device 210. For example, the implantable device 202 can transmit information to the wearable device 210 associated with one or more of the signal delivery parameters of the modulation signal being applied to the patient. In these and other embodiments, one or more of the implantable devices 202 can include a hermetic package or housing configured such that the implantable device(s) 202 can be implanted within a patient.

In some embodiments, one or more of the implantable devices 202 are passive devices that do not include an onboard pulse generator configured to generate modulation signals. Instead, the passive implantable device can wirelessly receive a power signal from the wearable device 210 and transmit the received power signal to the wearer via the electrodes. The passive implantable device may condition or otherwise process the received power signal, but does not use the received power signal to power an onboard pulse generator.

FIG. 2B is a side view the wearable device 210. In the illustrated embodiment, the wearable device 210 is configured to be worn on or near an underside of the patient's jaw J. The wearable device 210 can be configured to transmit energy E, such as electromagnetic radiation in the form power and/or modulation signals, via one or more of the power transmission devices 212a, b to an implantable device 202 positioned at least proximate to the hypoglossal nerve HGN and/or one or more distal branches DB thereof.

FIG. 2C is another side view of the wearable device 210. In the illustrated embodiment, the wearable device 210 is configured to be worn on or at least partially around the wearer's neck N. The wearable device 210 can be configured to transmit energy E, such as electromagnetic radiation in the form power and/or modulation signals via one or more of the power transmission devices 212a, b to an implantable device 202 positioned at least proximate to the ansa cervicalis AC.

Both FIGS. 2B and 2C illustrate embodiments in which the wearable device 210 is configured to transmit power to one target location within the patient P, i.e., at least proximate to the hypoglossal nerve HGN in FIG. 2B and at least proximate to the ansa cervicalis nerve AC in FIG. 2C. In other embodiments, the wearable device 210 can be configured to transmit power to multiple target locations within the patient, such as at least proximate to the hypoglossal nerve HGN (e.g., as in FIG. 2B) and at least proximate to the ansa cervicalis nerve AC (e.g., as in FIG. 2C). In these and/or other embodiments, the wearable device 210 can include one or more power transmission devices configured to transmit power to multiple target locations, or a plurality of power transmission devices with one or more of the power transmission devices configured to transmit power to at least one of the multiple target locations.

FIG. 3A is a perspective view of a wearable device 310 configured in accordance with embodiments of the present technology. The wearable device 310 can include a first or primary support member 314 and a second or secondary support member 316. The primary support member 314 can extend between and/or include a first primary end portion 320a and a second primary end portion 320b. The primary support member 314 can have an open or torc ring shape in which the first primary end portion 320a and the second primary end portion 320b define a primary opening or gap 330 therebetween. The gap 330 can be positioned on an anterior side of the neck N. This anterior positioning of the gap 330 can allow a wearer to use the gap 330 to register the wearable device 310 with the wearer's trachea and/or one or more other anatomical features at an anterior side of the wearer's neck N to, e.g., rotationally align the wearable device 310 relative to the wearer's neck N when donning the wearable device 310. Additionally, or alternatively, the gap 330 can enable the wearable device 310 to sit comfortably around the wearer's neck N without, or substantially without, restricting caudal traction and/or other movement of and/or within the neck N. The primary support member 314 can include Nitinol, silicone, and/or one or more other flexible materials that allow the primary gap 330 to increase and/or decrease in size to fit the primary support member 314 at least partially around the neck N.

The secondary support member 316 can be at least generally similar or identical in structure and/or function to the primary support member 314. For example, the secondary support member 316 can extend between and/or include a first secondary end portion 322a and a second secondary end portion 322b. The secondary support member 316 can have an open or torc ring shape in which the first secondary end portion 322a and the second secondary end portion 322b define a secondary opening or gap 332 therebetween. The secondary support member 316 can include Nitinol, silicone, and/or one or more other flexible materials that allow the secondary gap 332 to increase and/or decrease in size to fit the secondary support member 316 at least partially around the neck N.

In some embodiments, the primary support member 314 or the secondary support member 316 are omitted. In these and/or other embodiments, the wearable device 310 can include one or more additional support members. In the illustrated embodiment, for example, the wearable device 310 includes a third or tertiary support member 318 positioned between the primary support member 314 and the secondary support member 316, e.g., superior to the primary support member 314 and inferior to the secondary support member 316. The tertiary support member 318 can be at least generally similar or identical in structure and/or function to the primary support member 314 and/or the secondary support member 316. For example, the tertiary support member 318 can extend between and/or include a first tertiary end portion 324a and a second tertiary end portion 324b. The tertiary support member 318 can have an open or torc ring shape in which the first tertiary end portion 324a and the second tertiary end portion 324b define a tertiary opening or gap 334 therebetween. The tertiary support member 318 can include Nitinol, silicone, and/or one or more other flexible materials, such as one or more polymers, meshes, fabrics, and/or combinations thereof, that allow the tertiary gap 334 to increase and/or decrease in size to fit the tertiary support member 318 at least partially around the neck N.

When seated around respective portions of the neck N, each of the support members 314, 316, 318 is expected to move at least generally with the respective neck portion, without or generally without slipping and/or restricting the wearer's ability to move their neck N. For example, the primary support member 314 can be worn around or at least proximate to the portion of the neck N defined by first plane 100a (FIG. 1C) and the secondary support member 316 can be worn around or at least proximate to the portion of the neck N defined by the second plane 100 b (FIG. 1C). As the wearer moves these planes 100a, b of their neck N, the primary support member 314 and/or the secondary support member 316 can move with the neck N and/or relative to one another, tracking the neck's movement within and/or relative to these respective planes 100a, b (FIG. 1C). That is, the primary support member 314 can be seated around and move (e.g., rotate, pitch, yaw, roll, etc.) with a first portion of the neck moving within and/or relative to the first plane 100a without, or generally without, moving and/or changing orientation relative to the first portion of the neck N, and/or the secondary support member 316 can be seated around and move (e.g., rotate, pitch, yaw, roll, etc.) with a second portion of the neck moving within and/or relative to the second plane 100b without, or substantially without, moving and/or changing orientation relative to the second portion of the neck N. For example, each of the end portions 320, 322, 324 can contact a respective region of the wearer's neck N when the wearable device 310 is worn thereon. When the wearer moves their neck N, the support members 314, 316, 318 can move relative to one another such that the end portions 320, 322, 324 stay in contact with, or at least generally stay in contact with, the respective regions of the wearer's neck N, without or generally without restricting the wearer's ability to move their neck N. For example, in response to a movement of the wearer's head H and/or neck N (FIG. 1C), individual ones of the support members 314, 316, 318 can undergo up to 50%, 60%, 70%, 80%, 90%, or 100% of the movement experienced by the corresponding portion about which the support member 314, 316, 318 is seated. Accordingly, in at least some embodiments, such as when the secondary support member 316 contacts a portion of the neck N superior to the primary support member 314, the secondary support member 316 can rotate independently of the primary support member 314. This is described in greater detail below with reference to FIGS. 3B-3F.

The support members 314, 316, 318 can be coupled to one another via one or more connectors. In the illustrated embodiment, for example, one or more primary connectors 326 (individually identified as a first primary connector 326a and a second primary connector 326b) couple the primary support member 314 with the tertiary support member 318, and one or more secondary connectors 328 (individually identified as a first secondary connector 328a and a second secondary connector 328b) couple the tertiary support member 318 with the secondary support member 316. Although the primary connectors 326 are coupled between the first end portions 320a, 324a and between the second end portions 320b, 324b of the support members 314, 318 in FIG. 3A, in other embodiments the primary connectors 326 can be coupled between other suitable portions of the support members 314, 318.

For embodiments in which the tertiary support member 318 is omitted, the secondary connectors 328 can also be omitted and the primary connector 326 can couple the primary support member 314 with the secondary support member 316. The connectors 326, 328 can be configured to allow the support members 314, 316, 318 to move (e.g., inferior, superiorly, anteriorly, posteriorly, yaw, pitch, roll, and/or combinations thereof) relative to one another as the patient moves their neck N. In the illustrated embodiment, for example, individual ones of the connectors 326, 328 are curved, arcuate, and/or looped, e.g., such that they have a length greater than a distance between immediately adjacent ones of the support member 314, 316. 318. This, in turn, can allow the support members 314, 316, 318 to move relative to one other without, or at least substantially without, interference from the connectors 326, 328. Increasing the length of the connectors can, in turn, increase the degree of rotation they permit the support members 314, 316, 318. Additionally, or alternatively, the connectors 326, 328, can include Nitinol, silicone, and/or one or more other flexible materials. In these and/or other embodiments, the connectors 326, 328 can include one or more springs, sliding rods, joints, kinematic chains, and/or one or more other suitable connectors. In at least some embodiments, the primary connectors 326 can be in compression between the primary and tertiary support members 314, 318 and/or the secondary connectors 328 can be in compression between the secondary and tertiary support members 316, 318, e.g., configured to apply an opposing force between the support members 314, 316, 318 to bias the support members 314, 316, 318 away from one another along an inferior-superior or cephalad-caudal axis which, in turn, can help space the support members 314, 316, 318 along the neck and/or seat the support members 314, 316, 318 around respective portions of the patient's neck N. The location along the support members 314, 316, 318 to which the connectors 326, 328 are coupled can affect the anterior/posterior and/or lateral distribution of the biasing force on the support members 314, 316, 318 from the connectors 326, 328. For connectors 326, 328 of a given stiffness (e.g., spring constant), the force applied by the connectors 326, 328 to the support members 314, 316, 318 can vary based on a length of the connectors 326, 328, e.g., with longer connectors 326, 328 being more flexible and/or providing less resistance to movement than shorter connectors 326, 328. In some embodiments, the connectors 326, 328 can be configured to apply the greater force at one or more circumferential location of increasing size, e.g., left to right near the base of the neck N and/or anterior/posterior at the chin C.

The wearable device 310 can be configured to transmit power (e.g., wirelessly) to one or more implantable devices (e.g., the implantable devices 202 of FIGS. 2A-2C). For example, the wearable device 310 can include one or more power transmission devices 312 (shown schematically in dashed lines, and individually identified as first through sixth power transmission devices 312a-f, respectively) and/or an electronics housing 336. In some embodiments, the wearable device 310 can include as many power transmission devices 312 as there as implanted devices positioned within the patient's neck N (e.g., one power transmission device for one implantable devices, two power transmission devices for two implantable devices, etc.). In other embodiments, the wearable device 310 can include a number of power transmission devices 312 that is greater or less than the number of implantable devices (e.g., one power transmission device for two implantable devices, two power transmission devices for one implantable devices, etc.). In some embodiments, having more power transmission devices 312 than implantable devices 202 enables the wearable device 310 to dynamically select one of the power transmission devices 312, or a subset of the power transmission devices 312, to provide power to a given implantable device 202. For example, the power transmission device(s) 312 can be selected based at least in part on which of the power transmission device(s) 312 is closest to the implantable device 202 and/or has the best coupling with a power receiving component carried by the implantable device 202. The wearable device 310 can accordingly compensate for migration and/or other movement of the power transmission device(s) 312 relative to the patient and/or the implantable device 202. For example, during sleep, patient movement can cause the wearable device 310 to move in relation to the patient by a few centimeters. Since migration of the wearable device 310 can disrupt or otherwise effect power transmission to the implantable device 202, having multiple power transmission devices 312 at various locations on the wearable device 310 enables the wearable device 310 to continuously provide therapy even after the wearable device 310 migrates. This is achieved, for example, by dynamically switching to the power transmission device(s) that provides the best coupling with the implantable device(s), which is generally the power deliver device that is closest to the implantable device at any given moment.

Each of the power transmission devices 312 can be coupled to one of the support members 314, 316, 318, and the support members 314, 316, 318 can be configured to hold the respective power transmission devices 312 against the neck with sufficient force (e.g., a sufficient uniform circumferential compressive force, including between 0.01 pounds per square inch (PSI) and 1 PSI ) to maintain a position of the power transmission device 312 relative to one or more devices implanted within the patient while also being comfortable for the patient (e.g., without or substantially without skin blanching or contact pressure that could cause pressure or shear on the skin to irritate it). The illustrated embodiment of the wearable device 310 includes six power transmission devices 312a-f, with each coupled to one of the end portions 320a, 320b, 322a, 322b, 324a, 324b. As noted above, one or more of the support members 314, 316, 318 can undergo up to 50%, 60%, 70%, 80%, 90%, or 100% of the movement experienced by the corresponding portion of the neck N about which the support member 314, 316, 318 is seated. Accordingly, one or more of the power transmission devices 312 can undergo and/or maintain contact with the neck N for up to 50%, 60%, 70%, 80%, 90%, or 100% of the movement experienced by the corresponding portion of the neck N about which the support member 314, 316, 318 is seated. Additionally, or alternatively, the wearable device 310 can include one or more power transmission devices 312 positioned between any two of the end portions 320a, 320b, 322a, 322b, 324a, 324b and/or at one or more other suitable locations. In these and/or other embodiments, the power transmission devices 312 can be positioned based, at least in part, on the target site within the patient at which one or more implanted devices are to be positioned.

Although the power transmission devices 312 are described as being configured to transmit power to one or more implantable devices, in some embodiments one or more of the power transmission devices 312 are configured to provide transcutaneous electrical modulation to one or more target locations within the patient's neck N. For example, at least one of the power transmission devices 312 can be positioned to transcutaneously modulate the ansa cervicalis and/or one or more of the muscles innervated thereby.

The electronics housing 336 can contain one or more batteries, processors, computer memory, and/or other components operable (e.g., independently or in combination) to cause individual power transmission devices 312 to transmit power. In some embodiments, the electronics housing 336 can include and/or be operably coupled to one or more feedback components, such as speakers, lights, haptic engines, etc., configured to provide feedback (e.g., audio feedback, visual feedback, haptic feedback, etc.) to the user. The feedback, for example, can help the user don, activate, deactivate, and/or otherwise operate the wearable device 310. The electronics housing 336 can be coupled to one or more of the support members 314, 316, 318, e.g., to be positioned on a posterior side of the neck N when the wearable device 310 is worn (e.g., to reduce or prevent interfering with caudal traction and/or other movement at or near an anterior side of the neck N during treatment). The posterior side of the neck N can define a natural concavity and, accordingly, positioning the electronics housing 336 at or near this concavity is expected to reduce, or even prevent, patient discomfort at least when the patient is supine. Additionally, when the patient moves their head H and/or neck N, the posterior side of the neck N is expected to undergo less movement relative to the anterior side and, accordingly, positioning the electronics housing 336 at or near the posterior side of the neck N is expected to reduce, or even prevent, interference with the wearer's ability to freely move their neck N.

FIGS. 3B-3F are perspective views of the wearable device 310 showing representative movement of the wearable device 310 with the wearer's neck N. The power transmission devices 312 and the electronics housing 336 (FIG. 3A) are omitted from FIGS. 3B-3F for purposes of illustrative clarity. FIG. 3B shows that, in response to a forward tilt of the neck N, the first and second tertiary end portions 324a, b of the tertiary support member 318 have moved closer to the first and second primary end portions 320a, b of the primary support member 314, and that the primary connectors 326 have been deformed (e.g., relative to FIG. 3A) in response to this movement.

FIGS. 3C and 3D show that, in response to a right rotation of the neck N, the support members 314, 316, 318 rotate with the neck N, with the secondary support member 316 rotating a greater degree than the tertiary support member 318 and/or the primary support member 314. FIG. 3D additionally shows that, after the right rotation of the neck N, the support members 314, 316, 318 can move with the neck N when the wearer elevates their chin C.

FIGS. 3E and 3F shows that, in response to a left rotation of the neck N, the support members 314, 316, 318 rotate with the neck N, with the secondary support member 316 rotating a greater degree than the tertiary support member 318 and/or the primary support member 314. FIG. 3F additionally shows that, after the left rotation of the neck N, the support members 314, 316, 318 can move with the neck N when the wearer lowers their chin C. Referring to FIGS. 3B-3F, it will be appreciated that the movements shown and described are a representative subset of the possible movements and that the wearable device 310 is configured to track additional and/or other movements of the wearer's neck N.

In the embodiment illustrated in FIGS. 3A-3F the primary support member 314 is positioned inferior to the tertiary support member 318 and the secondary support member 316 is positioned superior to the tertiary support member 318. In other embodiments, such as shown in FIGS. 3G and 3H, the primary support member 314 can extend outwardly from the tertiary support member 318 in a first (e.g., inferior) direction and/or the secondary support member 316 can extend outwardly from the tertiary support member 318 in a second (e.g., superior) direction opposite the first direction. For example, the first and second primary end portions 320a, b of the primary support member 314 can be coupled to the tertiary support member 318 so that the primary support member 314 defines a loop or other arcuate structure extending (inferiorly) from the tertiary support member 318. Additionally, or alternatively, the first and second secondary end portion 322a, b of the secondary support member 316 can be coupled to the tertiary support member 318 so that the second support member 316 defines a loop or other arcuate structure extending (superiorly) from the tertiary support member 318. In these and/or other embodiments, the primary support member 314 and/or the secondary support member 316 can be flexible and configured to bend or deform to track movement of the patient's N, e.g., without including the connectors 326, 328. The power transmission devices 312 (FIG. 3H) can be coupled to various locations on the primary, secondary, or tertiary support members 314, 316, 318. The electronics housing 336 can be coupled to a posterior portion of the tertiary support member 318, e.g., to be positioned on a posterior side of the patient's neck N when worn.

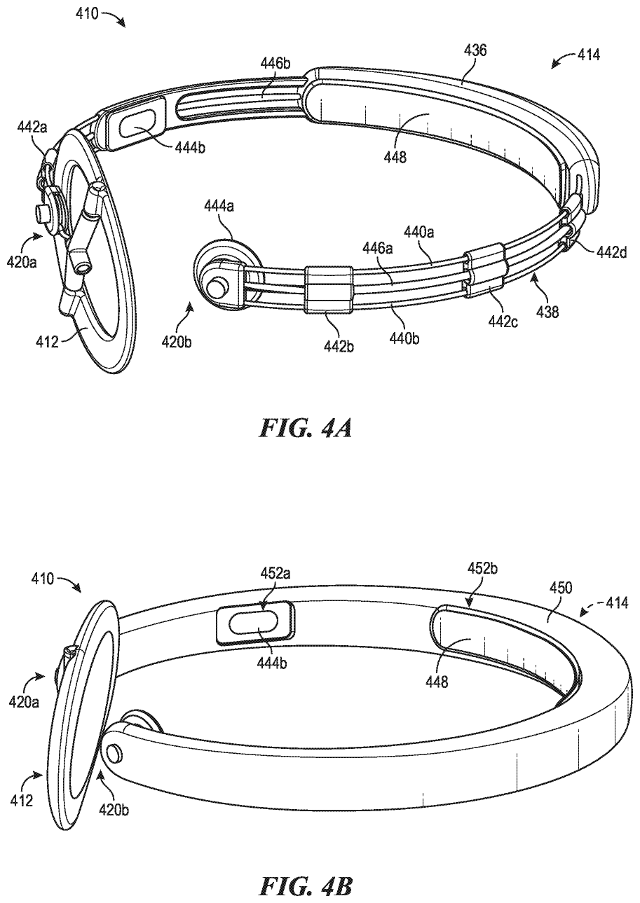

FIGS. 4A and 4B are perspective views of another wearable device 410 configured in accordance with embodiments of the present technology. Referring first to FIG. 4A, the wearable device 410 can include features that are at least generally similar or identical in structure and/or function to the wearable device 310 of FIGS. 3A-3F. For example, the wearable device 410 can include a support member 414 extending between or including a first end portion 420a and a second end portion 420b, a power transmission device 412, and/or an electronics housing 436 that can be at least generally similar or identical in structure and/or function to the correspondingly named and/or numbered feature of the wearable device 310 of FIGS. 3A-3F. The support member 414 can be positioned at least partially around a user's neck, e.g., to position the power transmission device 412 to transmit power to one or more implantable devices positioned within the user's neck.

In the illustrated embodiment, the support member 414 further includes a wire body 438 shaped to form an open ring/torc shape. The end portions 420 of the wire body 438 can be free ends or portions at which the wire body 438 is curved over itself, e.g., to form a superior portion 440a and an inferior portion 440b of the wire body 438. In some embodiments, rather than folding the wire body 438 to form superior and inferior portions 440a, b, two wires can be used with one positioned superior to the other. In some embodiments, one or more clips or retainers 442 (individually identified as first through fourth retainers 442a-d, respectively) can be coupled to the wire body 438, e.g., between the superior and inferior portions 440a, b. The retainers 442 can slidably receive the wire body 438 to space apart the superior and inferior portions 440a, b but otherwise allow the wire body 438 to move (e.g., laterally) relative to one or more of the retainers 442. In some embodiments, the retainers 442 can be configured as primary skin-contacting element and/or positioned to avoid pressure-susceptible areas of the patient's anatomy, such as tissues overlying the carotid artery.

In some embodiments, the wearable device 410 can include one or more sensors 444 (individually identified as a first sensor 444a and a second sensor 444b). In the illustrated embodiment the first sensor 444a is a microphone coupled to the second end portion 420b and the second sensor 444b is a photoplethysmography (PPG) sensor coupled to the electronics housing 436. In other embodiments, the sensors 444 can have other suitable locations and/or include other suitable sensors, including one or more temperature sensors, heart rate sensors, electromyography (EMG) sensors, accelerometers, contact sensors, acoustic sensors, pressure sensors, inclinometers, etc. One or more of the sensors 444 and/or the power transmission devices 412 can be electrically coupled to the electronics housing 436 and/or one or more components contained therein via one or more respective communication wires 446 (individually identified as a first communication wire 446a electrically coupled to the first sensor 444a and a second communication wire 446b electrically coupled to the power transmission device 412).

In some embodiments, the wearable device 410 can include one or more contact surfaces 448. While the other portions of the wearable device 410 may contact, and/or be configured to contact, the neck N when the wearable device 410 is worn, the contact surfaces 448 can be configured to have increased friction relative to all, or at least a subset, of these other portions of the wearable device 410. For example, at least one of the contact surfaces 448 can include silicone and/or one or more other materials configured to reduce to prevent movement of the wearable device 410 relative to the neck N and/or improve patient comfort (e.g., hypoallergenic, biocompatible, durable, etc.). Additionally, or alternatively, at least one of the contact surfaces 448 can be textured or include friction-increasing features, such as one or more ridges, adhesives, etc.

Referring to FIG. 4B, in some embodiments the wearable device 410 can include a covering 450 configured to extend at least partially or fully around all, or at least a subset, of the components of the wearable device 410, e.g., described previously with reference to FIG. 4A. For example, the support member 414 (FIG. 4A), the wire body 438, and the retainers 442 can be fully contained within the covering 450. In some embodiments, the covering 450 is removable for cleaning and/or to provide access to various components contained within the covering 450. Additionally, or alternatively, the covering 450 can define one or more openings 452 (individually identified as a first opening 452a and a second opening 452b), e.g., aligned with various components contained within the covering 450. In the illustrated embodiment, for example, the first opening 425a is aligned with the second sensor 444b, e.g., to allow the second sensor 444b to obtain readings from the patient when worn. The second opening 425b can be aligned with the contact surfaces 448.

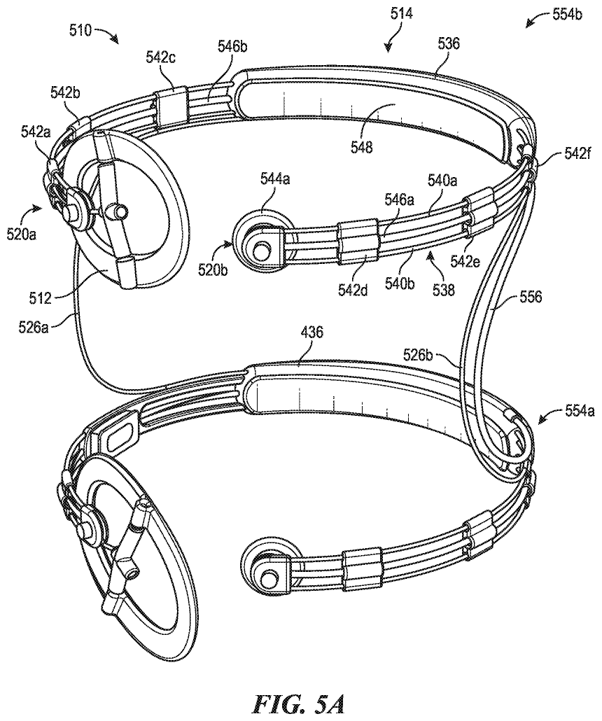

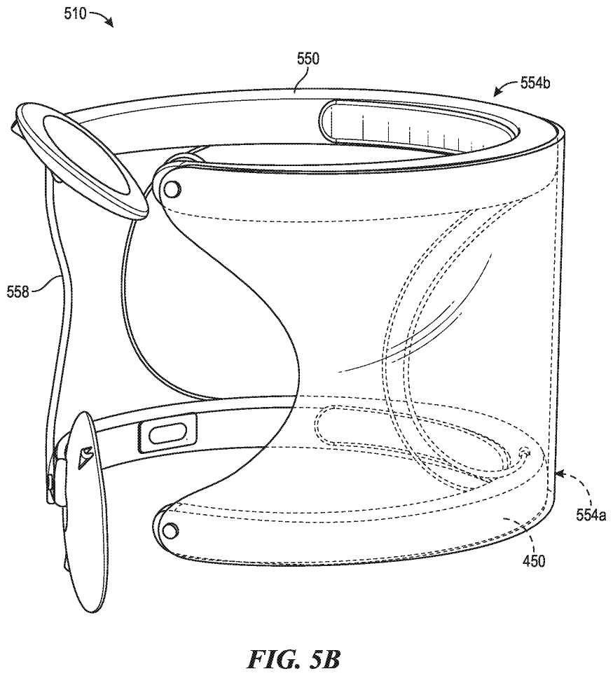

Referring to FIG. 5A, another embodiment of the wearable device 510 can include a primary or inferior portion 554a and a secondary or superior portion 554b. The primary portion 554a can be at least generally similar or identical in structure and/or function to the wearable device 410 of FIG. 4A. The secondary portion 554b can also be at least generally similar or identical in structure and/or function to the wearable device 410 of FIGS. 4A and 4B. For example, in the illustrated embodiment, the secondary portion 554b can include a support member 514 extending between or including a first end portion 520a and a second end portion 520b, a power transmission device 512, an electronics housing 536, a wire body 538, one or more retainers 542 (individually identified as first through sixth retainers 542a-f), one or more sensors 544 (individually identified as a first sensor 544a and a second sensor 544b), one or more communication wires 546 (individually identified as a first communication wire 546a and a second communication wire 546b), and one or more contact surfaces 548 that can be at least generally similar or identical in structure and/or function to the correspondingly named and/or numbered feature of the wearable device 410 of FIGS. 4A and 4B (e.g., the wire body 538 can be at least generally similar or identical in structure and/or function to the wire body 438, the electronics housing 536 can at least generally similar or identical in structure and/or function to the electronics housing 436, etc.)

The primary portion 554a and the secondary portion 554b can be coupled to one another by one or more connectors 526 (individually identified as a first connector 526a and a second connector 526b). The connectors 526 can be at least generally similar or identical in structure and/or function to the primary connectors 326 and/or the secondary connectors 328 in FIGS. 3A-3F. In the illustrated embodiment, the connectors 526 extend between and/or are coupled to the electronics housing 436 in the primary portion 554a and the electronics housing 536 in the secondary portion 554b. In these and/or other embodiments, the wearable device 510 can include one or more connectors 526 coupled to the primary portion 554a and/or the secondary portion 554b at other locations, such as the wire body 438, the wire body 538, and/or at any of the other features of the wearable device 510 described previously herein.

In addition to being mechanically coupled (e.g., via the connectors 526), in some embodiments the primary portion 554a and the secondary portion 554b can be communicatively and/or electrically coupled. For example, in the illustrated embodiment the electronics housing 436 in the primary portion 554a is coupled to the electronics housing 536 in the secondary portion 554b, e.g., via one or more wires 556. The wires 556 can allow the electronics housings 436, 536 to communicate with one another, e.g., to provide power from one electronics housing to the other electronics housing, coordinate signal sensing, coordinate modulation signal delivery, etc. In these and/or other embodiments, the electronics housing 436, 536 can each include a wireless transceiver and/or other suitable communication component configured to communicatively and/or electrically couple the electronics housings 436, 536 and perform all, or at least a subset, of the above-noted functions wirelessly.

The wearable device 510 can be positioned at least partially around a user's neck. The primary portion 554a can be positioned as described previously with reference to FIG. 4A, e.g., to transmit power to one or more implantable devices positioned within the user's neck. The secondary portion 554b can be positioned superior to the primary portion 554a, e.g., so that the power transmission device 512 is positioned to transmit power to one or more implantable devices positioned superior to the primary portion 554a. For example, the power transmission device 512 can be positioned to transmit power to one or more implantable devices positioned at least proximate to an oral cavity and/or throat of the user. When worn, the primary portion 554a and the second portion 554b can move and/or rotate with the neck and/or relative to one another, as described previously with reference to the support members 314, 316, 318 of FIGS. 3A-3F.

Referring to FIG. 5B, the primary portion 554a can include the covering 450 described previously with reference to FIG. 4B. Additionally, or alternatively, the secondary portion 554b can include a covering 550 at least generally similar or identical in structure and/or function to the covering 450. In these and/or other embodiments, the wearable device 510 can include an overall or outer cover 558 configured to extend around all, or at least a portion, of the wearable device 510. In the illustrated embodiment, for example, the outer cover 558 is removably coupled to exterior surfaces of the primary portion 554a and/or the secondary portion 554b.

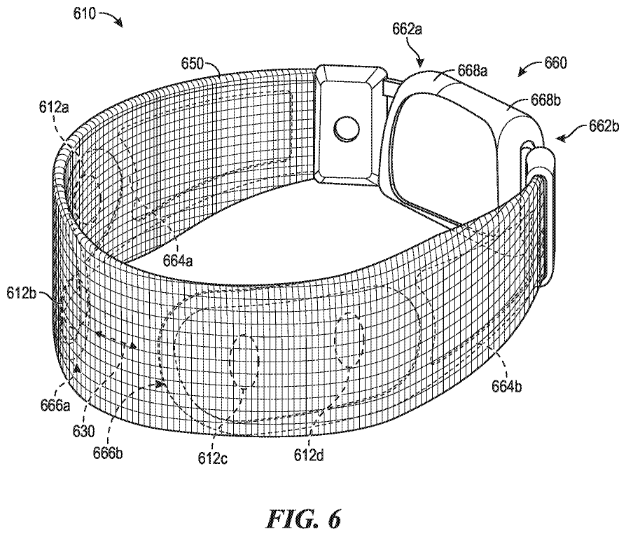

FIG. 6 is a perspective view of another wearable device 610 configured in accordance with embodiments of the present technology. At least some elements of the wearable device 610 can be at least generally similar or identical in structure and/or function to any of the wearable devices described above with reference to FIGS. 2A-5B. For example, the wearable device 610 can be configured to transmit power to one or more implantable devices positioned within a patient, as described in greater detail below.

The wearable device 610 can include a body 660, a first support arm 664a, and a second support arm 664b. The body 660 can include a first side portion 662a and a second side portion 662b opposite the first side portion 662a. The first support arm 664a can be coupled to and/or extend outwardly from the first side portion 662a of the body 660 to define a first end portion 666a. The second support arm 664b can be coupled to and/or extend outwardly from the second side portion 662b of the body 660 to define a second end portion 666b. The first end portion 666a of the first support arm 664a and the second end portion 666b of the second support arm 664b can define a gap 630 therebetween. The gap 630 can be at least generally similar or identical to the gap 330 described previously with reference to FIG. 3A. For example, the gap 630 can increase and/or decrease in size to fit the wearable device 610 at least partially around the neck N.

In some embodiments the body 660 is or can include a buckle, clasp, magnets, snaps, Velcro®, buttons, hooks, one or more breakaway or removable sections, and/or one or more other connection mechanisms configured to secure the first support arm 664a and the second support arm 664b relative to one another. In the illustrated embodiment, for example, the body 660 includes a first connector portion 668a and a second connector portion 668b configured to be releasably coupled to one another. The first connector portion 668a can include the first side portion 662a of the body 660 and be coupled to the first support arm 664a. The second connector portion 668b can include the second side portion 662b of the body 660 and can be coupled to the second support arm 664b.

The wearable device 610 can further include a covering 650, which can be at least generally similar or identical in structure and/or function to the covering 450 of FIG. 4B. The covering 650 can extend around all, or at least a portion, of the first support arm 664a and/or the second support arm 664b. In at least some embodiments, the covering 650 can span the gap 630 between the first and second end portions 666a, b. In these and/or other embodiments, the covering 650 can be at least generally elastic and/or otherwise configured to draw the first and second end portions 666a, b toward one another, e.g., to provide a comfortable and/or snug fit around the wearer's neck when the wearable device 610 is worn. Making the wearable device 610 more comfortable in this manner is expected to increase user compliance.

In some embodiments, the wearable device 610 can include one or more power transmission devices 612 (shown schematically in dashed lines, and individually identified as a first power transmission device 612a, a second power transmission device 612b, a third power transmission device 612c, and a fourth power transmission device 612d), that can be at least generally similar or identical in structure and/or function to the power transmission devices 212 of FIGS. 2A-2C. Each of the power transmission devices 612 can be coupled to first support arm 664a, the second support arm 664b, the body 660, and/or one or more other suitable portions of the wearable device 610. When the wearable device 610 is worn by the user, e.g., at least partially around the user's neck, at least one of the power transmission devices 612 can be positioned to transmit power to one or more implantable devices positioned within the wearer's neck. In some embodiments, the wearable device 610 can include an electronics housing 636, which can be at least generally similar or identical in structure and/or function to the electronics housing 336 of FIG. 3A. The electronics housing 636 can be coupled to the first support arm 664a, the second support arm 664b, the body 660, and/or one or more other suitable portions of the wearable device 610.

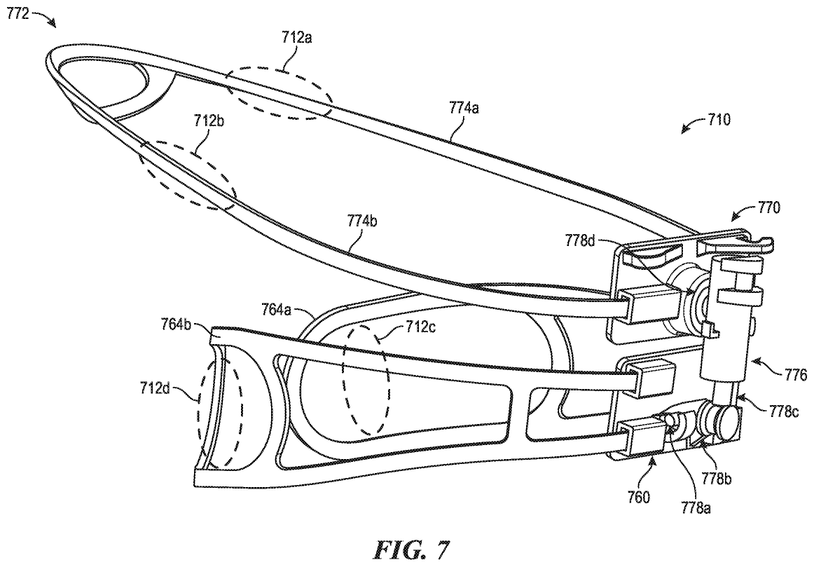

FIG. 7 is a perspective view of another wearable device 710 configured in accordance with embodiments of the present technology. At least some elements of the wearable device 710 can be at least generally similar or identical in structure and/or function to any of the wearable devices described above with reference to FIGS. 2A-6. For example, the wearable device can include a body 760 (e.g., a first body 760), a first support arm 764a, and a second support arm 764b. Additionally, the wearable device 710 can include a second body 770 and a jaw and/or chin support or contacting portion 772. The chin contacting portion 772 can be coupled to the second body 770 by one or more elongate chin contacting members or arms 774 (individually identified a chin contacting arm 774a and a second chin contacting arm 774b). The chin contacting arms 774 can, with the chin contacting portion 772, define a single-piece component, or one or more of the chin contacting arms 774 can be separate from and/or coupled to the chin contacting portion 772. One or more power transmission devices 712 (individually identified as a first power transmission device 712a, a second power transmission device 712b, a third power transmission device 712c, and a fourth power transmission device 712d) can be coupled to the chin contacting portion 772, one or more of the chin contacting arms 774, and/or one or more of the support arms 764.

When worn by a user, the support arms 764a, b can be positioned around the user's neck, e.g., to position at least one of the power transmission devices 712 to transmit power to one or more implantable devices positioned within the user's neck. The chin contacting portion 772 can support the wearer's chin while allowing the user to move their head relative to their neck without, or substantially without, interference from the wearable device 710, e.g., to position at least one of the power transmission devices 712 to transmit power to one or more implantable devices positioned at least proximate to an oral cavity of the patient. In at least some embodiments the second body 770 can be movably coupled to the first body 760 by an adjustable coupling or connector 776, configured to allow a user to move (e.g., freely move) their head and/or neck without, or generally without, interference from the wearable device 710. The adjustable connector 776 can include one or more joints 778 (individually identified as a first joint 778a, a second joint 778b, a third joint 778c, and a fourth joint 778d), each of which can define a corresponding axis of rotation and/or range of motion. The second joint 778b can be in series between the first and third joints 778a, c, and the third joint 778 c can be in series between the second and fourth joints 778b, d. The first joint 778a can define a first axis of rotation and the second joint 778b can define a second axis of rotation non-parallel (e.g., perpendicular) to the first axis of rotation. Additionally, or alternatively, the third joint 778c can define a third axis of rotation non-parallel (e.g., perpendicular) to the first axis of rotation and/or the second axis of rotation. In these and/or other embodiments, the fourth joint 778d can be a ball joint and/or otherwise have a greater range of motion than one or more of the first through third joints 778a-c. In other embodiments, one or more of the joints 778 can have one or more other suitable locations and/or arrangements.

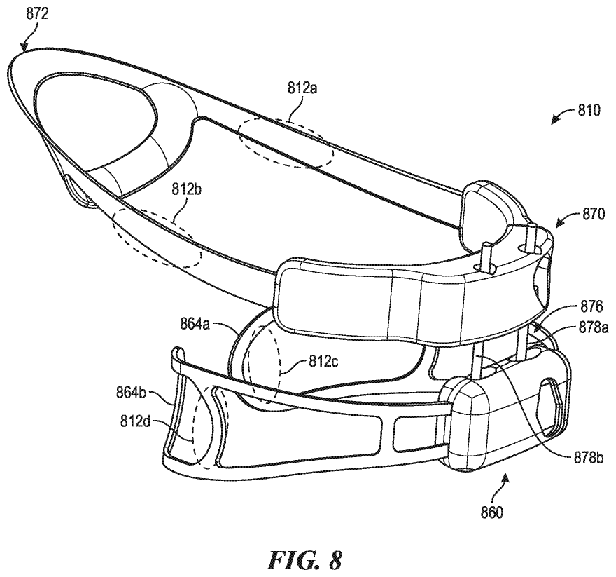

FIG. 8 is a perspective view of another wearable device 810 configured in accordance with embodiments of the present technology. At least some elements of the wearable device 810 can be at least generally similar or identical in structure and/or function to any of the wearable devices described above with reference to FIGS. 2A-7. For example, the wearable device 810 can include a first body 860, a first support arm 864a, a second support arm 864b, a second body 870, a jaw and/or chin contacting portion 872, and/or an adjustable coupling or connector 876 that can be at least generally similar or identical in structure and/or function to the correspondingly named and/or numbered feature(s) in FIG. 6 and/or FIG. 7. However, instead of joints, the adjustable connector 876 can include one or more flexible members 878 (individually identified as a first flexible member 878a and a second flexible member 878b). The flexible members 878 can be configured to undergo elastic deformation, or at least substantially elastic deformation, to allow a user to move (e.g., freely move) their head and/or neck without, or substantially without, interference from the wearable device 810.

One or more power transmission devices 812 (individually identified as a first power transmission device 812a, a second power transmission device 812b, a third power transmission device 812c, and a fourth power transmission device 812d) can be coupled to the chin contacting portion 872, one or more of the support arms 864, etc., as described previously with reference to FIG. 7. Accordingly, at least one of the power transmission devices 812 can be positioned to transmit power to one or more implantable devices positioned within the user's neck and/or at least proximate to an oral cavity of the patient.

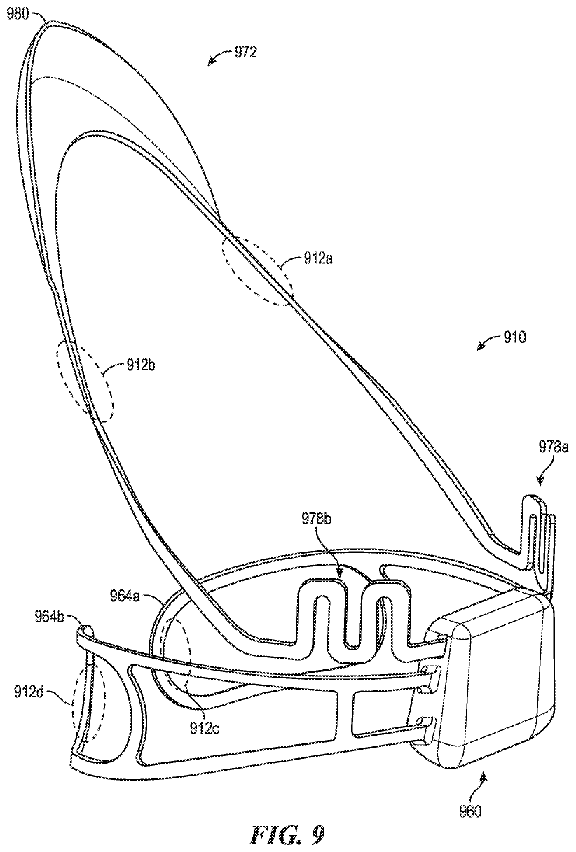

FIG. 9 is a perspective view of another wearable device 910 configured in accordance with embodiments of the present technology. At least some elements of the wearable device 910 can be at least generally similar or identical in structure and/or function to any of the wearable devices described above with reference to FIGS. 2A-8. For example, the wearable device 910 can include a body 960, a first support arm 964a, and a second support arm 964b, and/or a jaw and/or chin contacting portion 972, one or more of which can be at least generally similar or identical in structure and/or function the correspondingly named and/or numbered feature in FIG. 6 and/or FIG. 7. However, the chin contacting portion 972 is coupled to the body 960 (e.g., not another body that is coupled to the body 960) by one or more adjustable members 978 (individually identified as a first adjustable member 978a and a second adjustable member 978b). The adjustable members 978 can be configured to undergo elastic deformation, or at least substantially elastic deformation, to allow a user to move (e.g., freely move) their head and/or neck without, or substantially without, interference from the wearable device 910. In the illustrated embodiment, for example the adjustable members 978 have a z-wave and/or other zig-zag type of configuration. In these and/or other embodiments, the adjustable members 978 can have increased flexibility relative to one or more other portions of the wearable device. Additionally, in some embodiments, the chin contacting portion 972 can include a raised portion or lip 980 configured to cup or seat at least partially around the wearer's chin, e.g., to contact an anterior side of the wearer's chin.

One or more power transmission devices 912 (individually identified as a first power transmission device 912a, a second power transmission device 912b, a third power transmission device 912c, and a fourth power transmission device 912d) can be coupled to the chin contacting portion 972, one or more of the support arms 964, etc., as described previously with reference to FIG. 7. Accordingly, when the wearable device 910 is worn, the wearable device 910 can position at least one of the power transmission devices 912 to transmit power to one or more implantable devices positioned within the user's neck and/or at least proximate to an oral cavity of the patient.

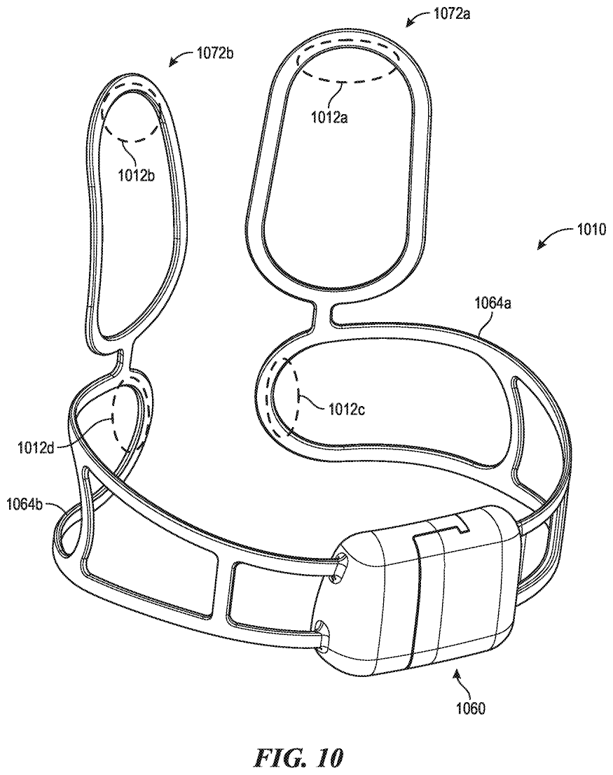

FIG. 10 is a perspective view of another wearable device 1010 configured in accordance with embodiments of the present technology. At least some elements of the wearable device 1010 can be at least generally similar or identical in structure and/or function to any of the wearable devices described above with reference to FIGS. 2A-9. For example, the wearable device 1010 can include a body 1060, a first support arm 1064a, and/or a second support arm 1064b that can be at least generally similar or identical in structure and/or function the correspondingly named and/or numbered feature in FIG. 6 and/or FIG. 7. Additionally, the wearable device 1010 can include one or more jaw and/or chin contacting portions 1072 (individually identified as a first chin contacting portion 1072a and a second chin contacting portion 1072b). Each of the chin contacting portions 1072 can be coupled to the first support arm 1064a, the second support arm 1064b, the body 1060, and/or to one or more other suitable locations on the wearable device 1010. In the illustrated embodiment, for example, the first chin contacting portion 1072a is coupled to the first support arm 1064a and the second chin contacting portion 1072b is coupled to the second support arm 1064b. When the wearable device 1010 is worn by a user, the support arms 1064a, b can be positioned at least partially around the user's neck, and the chin contacting portions 1072 can be deformed/deflected by the user's neck, chin, and/or jaw.

One or more power transmission devices 1012 (individually identified as a first power transmission device 1012a, a second power transmission device 1012b, a third power transmission device 1012c, and a fourth power transmission device 1012d) can be coupled to one or more of the chin contacting portions 1072, one or more of the support arms 1064, etc., as described previously with reference to FIG. 7. Accordingly, when the wearable device 1010 is worn, the wearable device 1010 can position at least one of the power transmission devices 1012 to transmit power to one or more implantable devices positioned within the user's neck and/or at least proximate to an oral cavity of the patient.

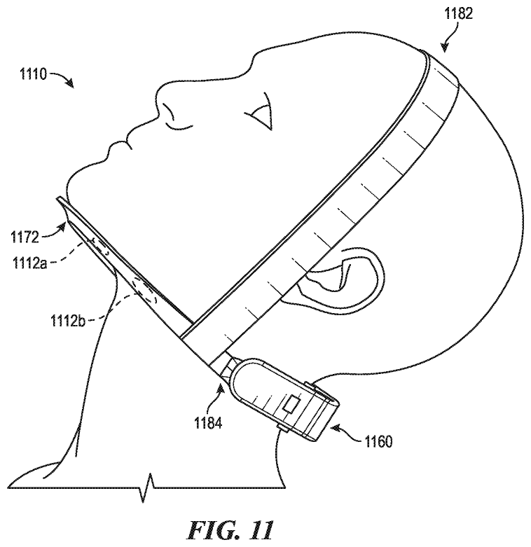

FIG. 11 is a perspective view of another wearable device 1110 configured in accordance with embodiments of the present technology. At least some elements of the wearable device 1110 can be at least generally similar or identical in structure and/or function to the wearable device 210 of FIGS. 2A-2C, the wearable device 310 of FIGS. 3A-3F, the wearable device 410 of FIGS. 4A and 4B, the wearable device 510 of FIGS. 5A and 5B, the wearable device 610 of FIG. 6, the wearable device 710 of FIG. 7, the wearable device 810 of FIG. 8, the wearable device 910 of FIG. 9, and/or the wearable device 1010 of FIG. 10. For example, the wearable device 1110 can include a body 1160, a jaw and/or chin contacting portion 1172, and/or one or more power transmission devices 1112 (individually identified as a first power transmission device 1112a and a second power transmission device 1112b) that can be at least generally similar or identical in structure and/or function the correspondingly named and/or numbered feature in FIGS. 6-10. The power transmission devices 1112 can be carried by the chin contacting portion 1172 and positioned to delivery power to one or more implantable devices positioned at least proximate to an oral cavity of the patient. However, the chin contacting portion 1172 in the illustrated embodiment is movably (e.g., rotatably) coupled to the body 1160 at a hinge or joint 1184 configured to allow the chin contacting portion 1172 to move relative to the body 1160, e.g., in response to movement of the user's head and/or neck. Additionally, the wearable device 1110 can include a head strap portion 1182 configured to extend at least partially around and/or over the user's head, e.g., to hold the chin contacting portion 1172 against an underside of the user's jaw. The head strap portion 1182 can be coupled to the chin contacting portion 1172 and/or one or more other suitable locations on the wearable device 1110.

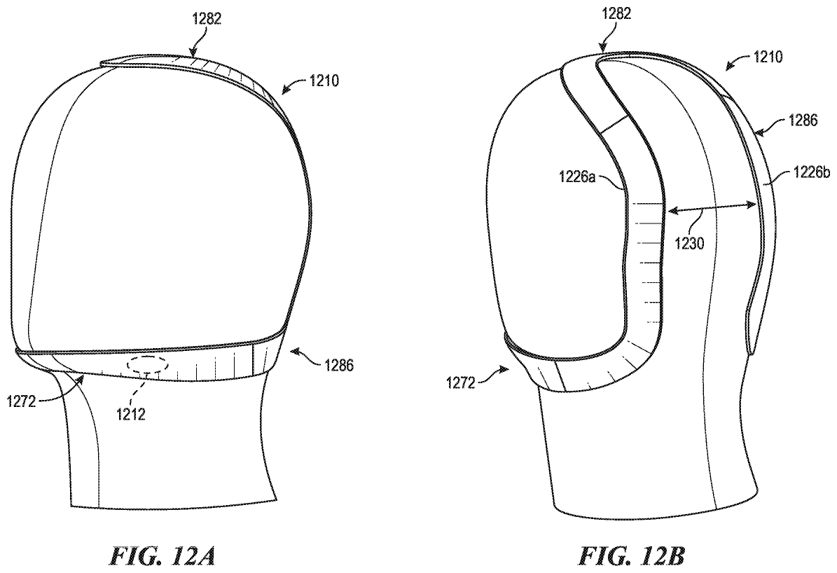

FIGS. 12A and 12B are front and rear perspective views, respectively, of another wearable device 1210 configured in accordance with embodiments of the present technology. At least some elements of the wearable device 1210 can be at least generally similar or identical in structure and/or function to any of the wearable devices described above with reference to FIGS. 2A-11. For example, the wearable device 1210 can include a jaw and/or chin contacting portion 1272, one or more power transmission devices 1212, and/or a head strap portion 1282 that can be at least generally similar or identical in structure and/or function the correspondingly named and/or numbered feature in FIGS. 6-10. However, instead of being mounted to a central body, the chin contacting portion 1272 and the head strap portion 1282 can be connected (e.g., directly) to one another by a connector portion 1286. Accordingly, the overall structure can have a continuous but non-planar elliptical shape. The connector portion 1286 can include one or more connectors 1226 (individually identified as a first connector 1226a and a second connector 1226b). In the illustrated embodiment, the first and second connectors 1226a, b define a gap 1230, e.g., positioned on a posterior side of the user's head when the wearable device is worn. The gap 1230 can extend at least partially or entirely between the first and second connectors 1226a, b. Additionally, or alternatively, the head strap portion 1282 and/or the chin contacting portion 1272 can be curved, arcuate, and/or at least generally U-shaped to at least partially define superior/upper and inferior/lower bounds, respectively, for the gap 1230. In some embodiments, the connector portion 1286 can be flexible and configured to draw the chin contacting portion 1272 and the head strap portion 1282 toward one another, e.g., to that the wearable device 1210 is held against superior and inferior surfaces of the user's head when worn.

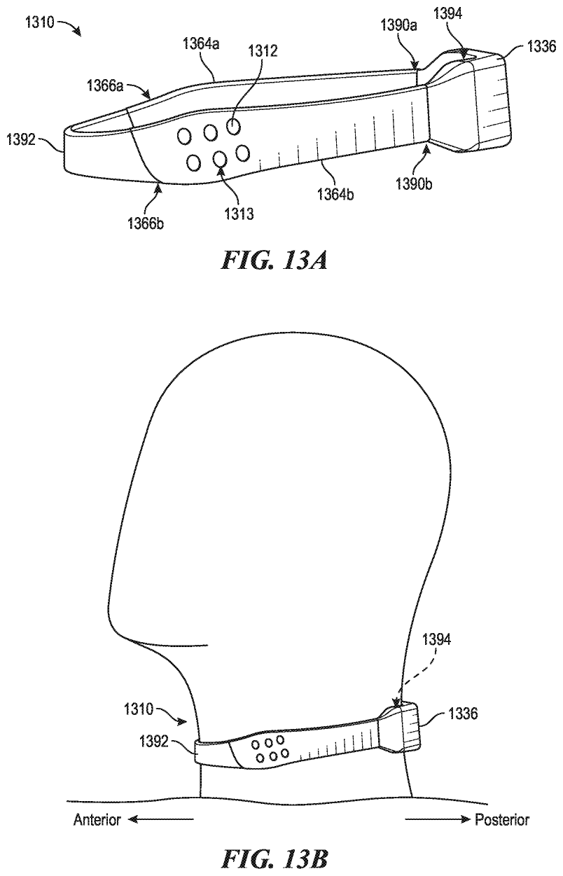

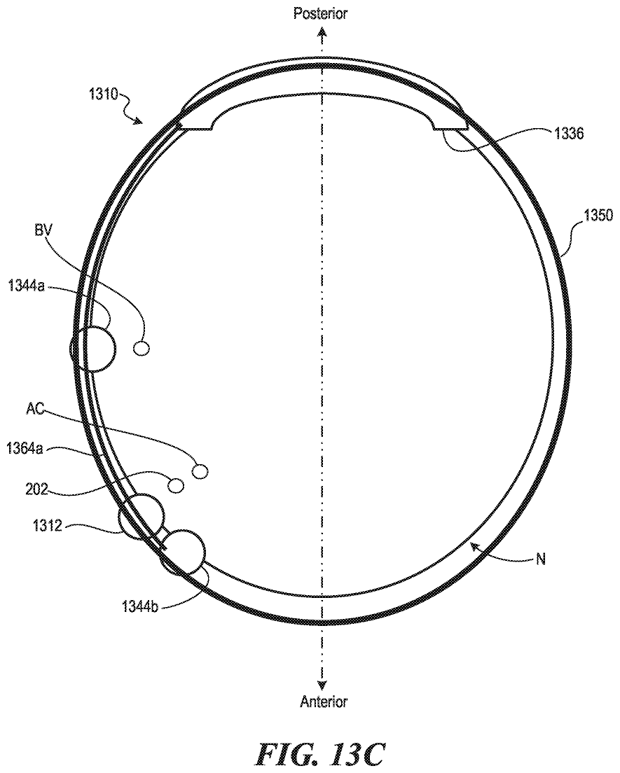

FIGS. 13A and 13B are perspective views of another wearable device 1310 configured in accordance with embodiments of the present technology. At least some elements of the wearable device 1310 can be at least generally similar or identical in structure and/or function to one or more elements of one or more of the wearable devices described above with reference to FIGS. 2A-12B. For example, the wearable device 1310 can include an electronics housing 1336, a first or right support member 1364a, and a second or left support member 1364b, which can be at least generally similar or identical in structure and/or function to the correspondingly named and/or numbered feature of one or more of the wearable devices of FIGS. 2A-12B.

The electronics housing 1336 can include an elongate body extending between a first or right end portion 1390a and a second or left end portion 1390b opposite the first end portion 1390a. In some embodiments, the electronics housing 1336 can be contoured, arcuate, and/or otherwise curved. For example, the electronics housing 1336 can define a concavity 1394 sized and/or shaped to rest flush, or at least partially flush, against the curvature of a posterior portion of a wearer's neck, such as shown in FIG. 13B. In some embodiments, the concavity 1394 can be sized and/or shaped to center the electronics housing 1336 at or about the wearer's midline. This, in turn, can help rotational alignment of the wearable device 1310 relative to the patient's neck when donning the wearable device 1310.

The first support member 1364a can be coupled or connected to the electronics housing 1336 at the first end portion 1390a, and extend outwardly and/or away from the electronics housing 1336 to define a first end portion 1366a opposite and/or spaced apart from the first end portion 1390a. The second support member 1364b can be coupled or connected to the electronics housing 1336 at the second end portion 1390b, and extend outwardly and/or away from the electronics housing 1336 to define a second end portion 1366b opposite and/or spaced apart from the second end portion 1390b. The first support member 1364a and/or the second support member 1364b can include a rigid or at least generally rigid arm. For example, the first and/or second support members 1364a, b can be configured to at least partially resist bending, flexing, twisting, deforming, etc., in response to externally applied forces. In other embodiments, the first support member 1364a and/or the second support member 1364 be can be and configured to undergo at least generally elastic deformation.

The wearable device 1310 can further include a connector portion 1392. The connector portion 1392 can extend between the first end portion 1366a of the first support member 1364a and the second end portion 1366b of the second support member 1364b. The connector portion 1392 can be at least generally elastic and/or otherwise configured to draw the first and second end portions 1366a, b toward one another to provide a comfortable and/or snug fit around the wearer's neck when the wearable device 1310 is worn. This, in turn, is expected to make the wearable device 1310 more comfortable and/or increase user compliance. In some embodiments, the first support member 1364a, the second support member 1364, and the connector portion 1392 define a flexible or expandable loop configured to be positioned around the wearer's neck. For example, each of the first support member 1364a, the second support member 1364, and the connector portion 1392 can be formed from one or more materials having elastic, or at least generally elastic, properties and configured to apply a compressive force to the wearer's neck sufficient to prevent, or at least generally prevent, the wearable device 1310 from moving relative to the wearer's head and/or neck.

The connector portion 1392 can be configured to contact an anterior portion of the wearer's neck (e.g., the trachea) when the wearable device 1310 is worn, such as shown in FIG. 13B. In some embodiments, for example, the connector portion 1392 can be configured to register with the wearer's trachea and/or one or more other anatomical features at an anterior side of the wearer's neck to rotationally align the wearable device 1310 relative to the wearer's neck when donning the wearable device 1310.

In some embodiments, the connector portion 1392 is configured to be sufficiently flexible to allow the wearer to don the wearable device 1310 by pulling the wearable device 1310 on over their head, e.g., without unbuckling, unclasping, or otherwise opening the wearable device 1310. In other embodiments, the wearable device 1310 can include one or more connector portions, such as the connector portions 668 described previously with reference to FIG. 6, that can be uncoupled to allow the wearable device 1310 to be placed around the wearer's neck and then re-coupled to secure the wearable device 1310 in place.

The wearable device 1310 can include one or more power transmission devices 1312. Individual power transmission devices 1312 can be coupled to the first support member 1364a, the second support member 1364b, the connector portion 1392, the electronics housing 1336, and/or other suitable portions of the wearable device 1310. In the illustrated embodiment, for example, the wearable device 1310 includes an array 1313 of power transmission devices 1312. The array 1313 can one or more rows and/or one or more columns of power transmission devices 1312. In the illustrated embodiment, the array 1313 includes two rows and three columns. In other embodiments, the array 1313 can include a greater or lesser number of rows and/or columns. In at least some embodiments, the wearable device 1310 can include more power transmission devices 1312 than implantable devices 202 (FIGS. 2A-2C). As described previously herein, having more power transmission devices 1312 than implantable devices 202 enables the wearable device 310 to dynamically select one of the power transmission devices 1312, or a subset of the power transmission devices 1312, to provide power to a given implantable device 202. The wearable device 1310 can accordingly compensate for migration and/or other movement of the power transmission device(s) 1312 relative to the patient and/or the implantable device 202.

FIG. 13C is a plan view of select features of the wearable device 1310, with other features omitted for the purpose of clarity. The wearable device 1310 can further include one or more sensors 1344 (individually identified as a first sensor 1344a and a second sensor 1344b). Individual power transmission devices 1312 and/or sensors 1344 can be coupled to the first support member 1364a at various locations, such as between the first support member 1364a and the patient's neck N, or with the support member 1364a between the neck N and the power transmission devices 1312 and/or the sensors 1344. Although the second support member 1364b is not shown in FIG. 13C, the second support member 1364b can also include one or more of the power transmission devices 1312 and/or one or more sensors 1344, e.g., in addition to or instead of the first support member 1364a. In some embodiments, the wearable device 1310 further includes a covering 1350 configured to extend at least partially or fully around all, or at least a subset, of the components (e.g., the first support member 1364a, the second support member 1364, the connector portion 1392, the electronics housing 1336, individual power transmission devices, individual sensors 1344, etc.) of the wearable device 1310.

The positions of one or more of the sensors 1344 along the first and/or second support members 1364a, b can be selected and/or adjusted based, at least in part, on the wearer's anatomy. For example, one or more of the sensors 1344 can be configured to assist the wearer in donning and/or aligning the wearable device 1310 about their neck N. In at least some embodiments, for example, the first sensor 1344a can include a PPG sensor configured to be aligned with a blood vessel BV within the wearer's neck N when the wearer is wearing the wearable device 1310 and the wearable device 1310 is positioned/aligned about the neck N as intended. Data from the PPG sensor can be used to detect the presence of the blood vessel BV, e.g., to confirm that the wearer is wearing the wearable device 1310 and that the wearable device 1310 is positioned/aligned about the neck N as intended. If the PPG sensor does not detect the blood vessel BV, the wearable device 1310 can provide feedback (e.g., audio, visual, haptic, etc.) to the user to indicate that the user should reorient the wearable device 1310. In another example, one or more of the sensors 1344, such as the second sensor 1344b, can include one or more capacitance sensors. The capacitance sensors can face inward, e.g., toward the wearer's neck N when the wearable device 110 is worn, such that the capacitance sensors can detect when they are in contact with the wearer's neck N. Accordingly, data from the capacitance sensors can be used to determine whether the wearer is wearing the wearable device 1310. If one or more of the capacitance sensors detect that they are not in contact with the wearer's neck, the wearable device 1310 can provide feedback (e.g., audio, visual, haptic, etc.) to the wearer to indicate that the wearer should reorient or adjust the position of the wearable device 1310. The sensors 1344 can be movably or removably coupled to the first support member 1364a and/or the second support member 1364b (e.g., via one or more screws, clips, press-fit couplings, etc.) to allow the clinician or other user to adjust the position of the sensors 1344 to better fit a given wearer's anatomy. For example, the clinician or other user can instruct the wearer to wear the wearable device 1310 for a trial period (e.g., one or more days or weeks) and reposition one or more of the sensors 1344 based, at least in part, on feedback from the wearer regarding their experience during the trial period, therapeutic efficacy, etc.

The positions of one or more of the power transmission devices 1312 can be selected and/or adjusted based, at least in part, on the wearer's anatomy and/or the target tissues selected to receive modulation signals. For example, FIG. 13C shows an implantable device 202 positioned within the neck N to deliver one or more modulation signals to the wearer's ansa cervicalis nerve N. The position of the power transmission device 1312 is selected to allow the power transmission device 1312 to transmit power to the implantable device 202, e.g., to power operation thereof. In some embodiments, the length of the first support member 1364a can be adjustable to allow a clinician or other user to position the power transmission device 1312 relative to the implantable device 202. In these and/or other embodiments, the power transmission device 1312 can be movably or removably coupled to the first support member 1364a (e.g., via one or more screws, clips, press-fit couplings, etc.) to allow the clinician or other user to move, remove, or otherwise adjust the power transmission device 1312, e.g., relative to the implantable device 202. For example, the clinician or other user can instruct the wearer to wear the wearable device 1310 for a trial period (e.g., one or more days or weeks) and reposition individual power transmission devices 1312 based, at least in part, on feedback from the wearer regarding their experience during the trial period, therapeutic efficacy, etc.

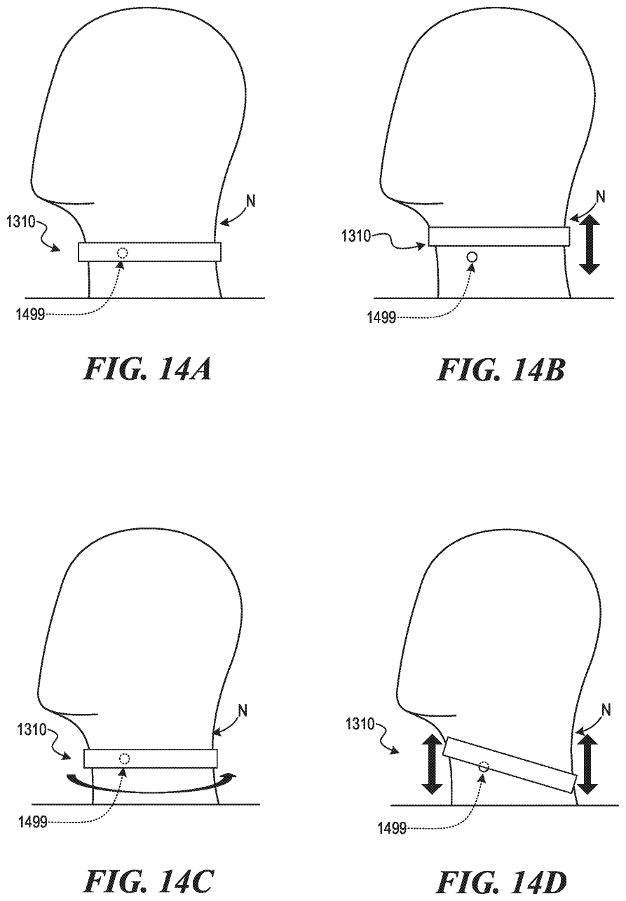

FIGS. 14A-14D are partially schematic perspective views of the wearable device 1310 positioned around the wearer's neck, in accordance with embodiments of the present technology. In FIG. 14A, the wearable device 1310 is rotationally aligned with the neck N and positioned to provide power to one or more implantable devices at a target point 1499 within the neck N. In FIG. 14B, however, the wearable device 1310 is longitudinally misplaced, e.g., positioned superior to the target point 1499. The longitudinal misplacement can be due, at least in part, to user error when donning the wearable device 1310 and/or migration during wear (e.g., while the wearer is asleep). In some embodiments, the wearable device 1310 is configured to detect this longitudinal misplacement, for example, using one or more of the sensors 1344 described with reference to FIG. 13C. The longitudinal misplacement can be due, at least in part, to user error when donning the wearable device 1310 and/or migration during wear (e.g., while the wearer is asleep). If the wearable device 1310 detects longitudinal misplacement, the wearable device 1310 can be configured to provide feedback (e.g., haptic feedback) to the user to indicate that the user should reposition the wearable device 1310. In some embodiments, the feedback is specific to the detected longitudinal misplacement, for example, a sequence of sounds and/or vibrations associated with positioning the wearable device 1310 higher or lower on the neck N. Additionally, or alternatively, the feedback can be configured to wake the wearer, e.g., to prompt the wearer to reposition the wearable device 1310. In these and/or other embodiments, the wearable device 1310 can be configured to stop transmitting power or turn off entirely in response to detected longitudinal misplacement.

In FIG. 14C, the wearable device 1310 is rotationally misaligned, e.g., positioned at a correct level on the neck N but rotated out of alignment with the neck N. The rotational misalignment can be due, at least in part, to user error when donning the wearable device 1310 and/or migration during wear (e.g., while the wearer is asleep). In some embodiments, the wearable device 1310 is configured to detect this rotational misplacement, for example, using one or more of the sensors 1344 described with reference to FIG. 13C. If the wearable device 1310 detects the rotational misplacement, the wearable device 1310 can be configured to provide feedback (e.g., haptic feedback) to the user to indicate that the user should reposition the wearable device 1310. In some embodiments, the feedback is specific to rotational misplacement, for example, a sequence of sounds and/or vibrations associated with rotating the wearable device 1310 left or right around the neck N. Additionally, or alternatively, the feedback can be configured to wake the wearer, e.g., to prompt the wearer to reposition the wearable device 1310. In these and/or other embodiments, the wearable device 1310 can be configured to stop transmitting power or turn off entirely in response to detected rotational misplacement.

In FIG. 14D, the wearable device 1310 is angularly misaligned, e.g., rotationally aligned with the neck N but tilted anterior-to-posterior such that an anterior side of the wearable device is positioned inferior or superior to a posterior side of the wearable device. The angular misalignment can be due, at least in part, to user error when donning the wearable device 1310 and/or migration during wear (e.g., while the wearer is asleep). In some embodiments, the wearable device 1310 is configured to detect this angular misalignment, for example, using one or more of the sensors 1344 described with reference to FIG. 13C. If the wearable device 1310 detects the angular misalignment, the wearable device 1310 can be configured to provide feedback (e.g., haptic feedback) to the user to indicate that the user should reposition the wearable device 1310. In some embodiments, the feedback is specific to angular misalignment, for example, a sequence of sounds and/or vibrations associated with tilting the wearable device 1310 to correct for angular misalignment. Additionally, or alternatively, the feedback can be configured to wake the wearer, e.g., to prompt the wearer to reposition the wearable device. In these and/or other embodiments, the wearable device 1310 can be configured to stop transmitting power or turn off entirely in response to detected angular misalignment.

It will be appreciated that FIGS. 14B-14D illustrate select examples of ways in which the wearable device 1310, and/or one or more of the other wearable devices described herein, can be misplaced or misaligned when worn, and that the wearable device 1310 and/or one or more of the other wearable devices described herein can be configured to detect and/or provide feedback regarding other misplacements and/or misalignments. For example, one or more of the wearable devices described herein can be configured to detect whether at least a portion of the wearable device is inside-out or inverted and, in response, provide feedback to the wearer to indicate that the wearer should reposition or reorient the wearable device to accordingly.

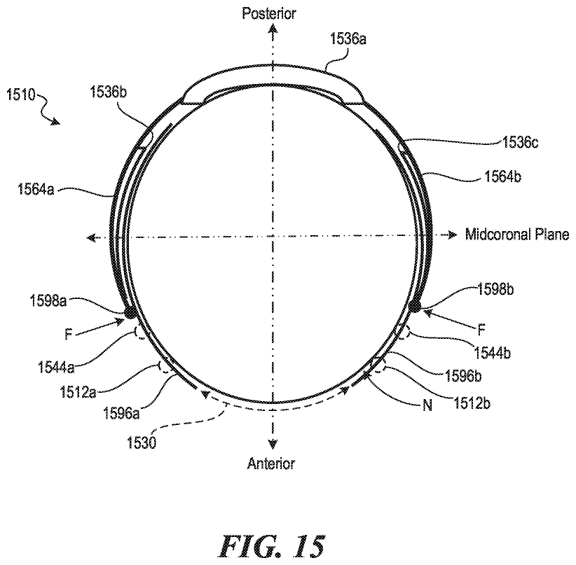

FIG. 15 is a top view of another wearable device 1510 configured in accordance with embodiments of the present technology. At least some elements of the wearable device 1510 can be at least generally similar or identical in structure and/or function to one or more elements of one or more of the wearable devices described herein with reference to at least FIGS. 2A-14D. For example, the wearable device 1510 can include an electronics housing 1536 (illustrated as including first, second, and third electronics housing portions 1536a-c), a first or right support member 1564a, and a second or left support member 1564b, each of which can be at least generally similar or identical in structure and/or function to the correspondingly named and/or numbered feature of one or more of the wearable devices of FIGS. 2A-14D.

The wearable device 1510 can further include one or more pads 1596 (individually identified as a first or right pad 1596a and a second or left pad 1596b). One or more of pads 1596 can include or carry one or more sensors 1544, b and/or power transmission devices 1512a, b (shown schematically in dashed line), such as any one or more of the sensors and/or power transmission devices described previously herein. The pads 1596 can define a gap 1530 therebetween configured to be aligned with an anterior portion of the wearer's neck N, e.g., at least generally similar or identical to the gap 330 described previously with reference to FIG. 3A.

Each of the pads 1596 can be coupled to a corresponding one of the support members 1564 via a pad connector or joint 1598 (individually identified as a first or right joint 1598a and a second or left joint 1598b). In the illustrated embodiment, for example, the first support member 1564a is coupled to the first pad 1596a via the first joint 1598a and the second support member 1564b is coupled to the second pad 1596b via the second joint 1598b. One or more of the joints 1598 can be rigid or flexible. The joints 1598 can include one or more hinges, fasteners, pins, ball joints, and/or other suitable joints.

The support members 1564a, b can be shaped to curved around the wearer's neck N and biased to exert a radially inward force F on the neck N when worn. For example, the support members 1564a, b can transfer the radially inward force F to the pads 1596 via the joints 1598, such that the pads 1596 press against the wearer's neck N. The joints 1598 can be positioned anterior to a midcoronal plane of the wearer such that at least a portion of the radially inward force F is directed posteriorly and/or toward the first or posterior electronics housing portion 1536a. This, in turn, can draw the first electronics housing portion 1536a anteriorly to retain the wearable device 1510 comfortably and securely about the wearer's neck N. In contrast, positioning the joints 1598 at or near the midcoronal plane may make the wearable device 1510 less stable and/or prone to falling off the wearer's neck N when disturbed (by, e.g., the wearer laying down, rolling over in their sleep, etc.). Thus, positioning the joints 1598 anterior to the midcoronal plane is expected to increase the stability of the wearable device 1510 and reduce the likelihood that the wearable device 1510 falls off the wearer's neck when disturbed.

In some embodiments, one or more of the pads 1596 can include silicone and/or be otherwise configured grip or frictively engage the wearer's neck N to, e.g., further reduce or prevent movement of the wearable device 1510 relative to the neck N. In some embodiments, all or at least a portion of one or more of the support members 1564 and/or one or more of the pads 1596 can be generally flexible and/or configured to conform to a contour of the wearer's neck N. In some embodiments, all or at least a portion of one or more of the support members 1564 and/or one or more of the pads 1596 can be at least generally rigid, under tension, and/or include multiple rigid segments configured to be drawn together via a spring force present therebetween so as to provide positive pressure against the skin.

In some embodiments, the electronics housing 1536 can be separated into portions 1536a-c that are distributed about the wearable device 1510 to improve weight balance. In the illustrated embodiment, for example, the electronics housing 1536 includes separate first, second, and third portions 1536a-c. The first electronics housing portion 1536a can be configured to be seated against a posterior side of the wearer's neck N and coupled to the first and second support members 1564a, b. The second electronics housing portion 1536 b can be configured to be positioned at least partially between the first support member 1564a and the first pad 1596a. The third electronics housing portion 1536c can be configured to be positioned at least partially between the second support member 1564b and the second pad 1596b. In other embodiments, the electronics housing 1536 can include more or fewer portions and/or one or more portions at other suitable locations.

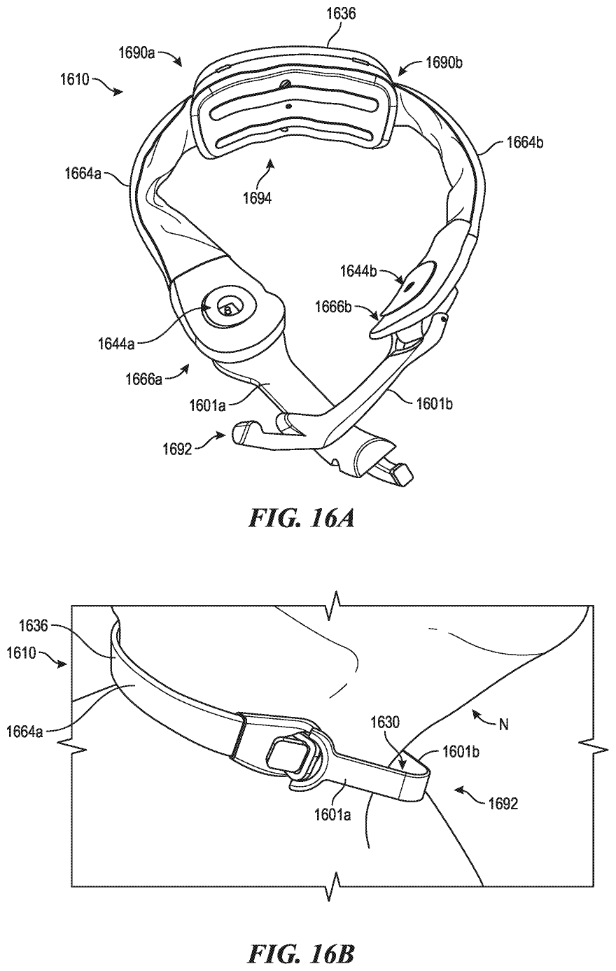

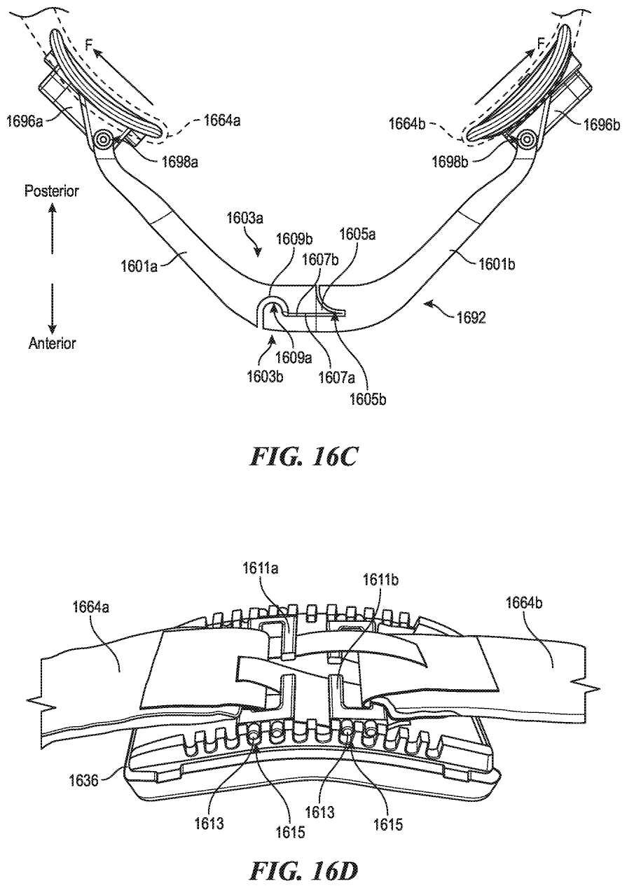

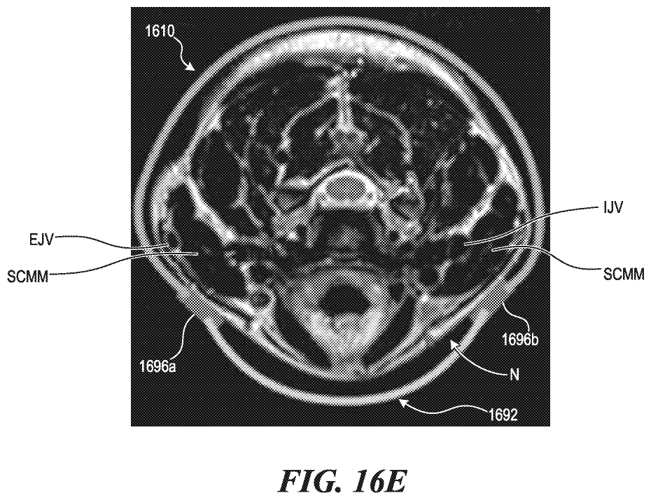

FIGS. 16A and 16B are perspective views of another wearable device 1610 configured in accordance with embodiments of the present technology. In FIG. 16A, the wearable device 1610 is shown in an unclasped state resting on a flat surface. In FIG. 16B, the wearable device 1610 is shown in a clasped state and positioned around a wearer's neck N. At least some elements of the wearable device 1610 can be at least generally similar or identical in structure and/or function to one or more elements of one or more of the wearable devices described herein with reference to at least FIGS. 2A-15. For example, the wearable device 1610 can include an electronics housing 1636, a first or right support member 1664a, and a second or left support member 1664b, each which can be at least generally similar or identical in structure and/or function to the correspondingly named and/or numbered feature of one or more of the wearable devices of FIGS. 2A-15.

Referring to FIG. 16A, the electronics housing 1636 can include an elongate body extending between a first or right end portion 1690a and a second or left end portion 1690b opposite the first end portion 1690a. In some embodiments, the electronics housing 1636 can be contoured, arcuate, and/or otherwise curved. For example, the electronics housing 1636 can define a concavity 1694 sized and/or shaped to rest flush, or at least partially flush, against the curvature of a posterior portion of a wearer's neck, such as shown in FIG. 16B. In some embodiments, the concavity 1694 can be sized and/or shaped to center the electronics housing 1636 at or about the wearer's midline. This, in turn, can help rotational alignment of the wearable device 1610 relative to the patient's neck when donning the wearable device 1610.

The first support member 1664a can be coupled or connected to the electronics housing 1636 at the first end portion 1690a and extend outwardly and/or away from the electronics housing 1636 to define a first end portion 1666a opposite and/or spaced apart from the first end portion 1690a. The second support member 1664b can be coupled or connected to the electronics housing 1636 at the second end portion 1690b and extend outwardly and/or away from the electronics housing 1636 to define a second end portion 1666b opposite and/or spaced apart from the second end portion 1690b. The first support member 1664a and/or the second support member 1664b can include one or more elastomeric materials, woven knit materials, rubbers, flexible non-stretchy fabrics, flexible materials, rigid materials, and/or combinations thereof. In the illustrated embodiment, for example, one or both of the first support member 1664a and the second support member 1664b can be configured to apply a compressive force to the wearer's neck sufficient to prevent, or at least generally prevent, the wearable device 1610 from moving relative to the wearer's head and/or neck. In other embodiments, the first support member 1664a and/or the second support member 1664b can include a rigid or at least generally rigid arm configured to at least partially resist bending, flexing, twisting, deforming, etc., in response to externally applied forces.

The wearable device 1610 can further include a bridge or connector portion 1692. The connector portion 1692 can extend between the first end portion 1666a of the first support member 1664a and the second end portion 1666b of the second support member 1664b. In some embodiments, the connector portion 1692 includes a first arm 1601a coupled to the first support member 1664a and a second arm 1601b coupled to the second support member 1664b. The wearer can couple (e.g., clasp) the first and second arms 1601a, b together, such as shown in FIG. 16B), to secure the wearable device 1610 around the wearer's neck N. The arms 1601a, b can be configured such that, when coupled together, the connector portion 1692 is spaced apart from the wearer's neck N, e.g., to define a gap 1630 (FIG. 16B) between the connector portion 1692 and an anterior side of the wearer's neck N. The wearer can uncouple or unclasp the first and second arms 1601a, b, such as shown in FIG. 16A, to facilitate removing the wearable device 1610 from their neck N. In some embodiments, the arms 1601a, b can be joined together to form a single-piece component and the wearable device 1610 can include one or more buckles, clasps, and/or other closure mechanisms configured to facilitate donning and removing the wearable device 1610 at a posterior or other portion of the wearable device 1610.

The wearable device 1610 can further include one or more sensors 1644 (individually identified as a first sensor 1644a and a second sensor 1644b). Individual sensors 1644 can be coupled to one or more of the support members 1664 at various locations. The positions of one or more of the sensors 1644 along the first and/or second support members 1664 a, b can be selected and/or adjusted based, at least in part, on the wearer's anatomy. For example, one or more of the sensors 1644 can be configured to assist the wearer in donning and/or aligning the wearable device 1610 about their neck N. In at least some embodiments, for example, the first sensor 1644a can include a PPG sensor and the second sensor 1644b can include a microphone and/or other audio sensor. Although not shown in FIG. 16A for simplicity, in some embodiments the electronics housing 1636 can include one or more of the sensors 1644, such as an accelerometer and/or other motion and/or orientation sensor.

FIG. 16C is a top view of the connector portion 1692 and the support members 1664 with other aspects of the wearable device 1610 omitted for illustrative clarity. Each of the arms 1601 can be coupled to a device housing 1696 (individually identified as first device housing 1696a and a second device housing 1696b) via a corresponding joint 1698 (individually identified as first joint 1698a and a second joint 1698b). Each joint 1698 can include a pin, a flexible material, a hinge, and/or one or more other suitable joints 1698. The location of the joints 1698 on each of the device housings 1696 can be adjustable to accommodate a wide range of neck sizes.

Each device housing 1696 can be generally rigid or generally flexible and can be configured to contain one or more functional components, such as one or more of the sensors 1644 described previously with reference to FIG. 16A and/or one or more power transmission devices. The first device housing 1696a can be coupled to and/or positioned at least partially within the first support member 1664a (shown in dashed line) and the second device housing 1696b can be coupled to and/or positioned at least partially within the second support member 1664b (shown in dashed line). One or more wires can extend through an interior of the support members 1664 to connect the sensors 1644 to the electronics housing 1636 and/or one or more of the components (e.g., battery, processor, etc.) contained therein. In some embodiments, one or more of the device housings 1696 can be configured to contact (e.g., directly contact) the wearer's neck N and/or can include a contact pad (e.g., silicone) configured to contact the wearer's neck N.

When the wearable device 1610 is worn, the support members 1664 can be configured to draw the device housings 1696 posteriorly and against the wearer's neck and the connector portion 1692 can be configured to balance or resist this posterior force so as to seat the wearable device 1610 comfortably and securely around the wearer's neck. Because the device housings 1696 can include the one or more sensors 1644, pressing the device housings 1696 firmly against the wearer's neck N is expected to improve the readings from one or more of the sensors 1644. In these and/or other embodiments, the connector portion 1692 can be configured to facilitate donning and removing the wearable device 1610. For example, each of the arms 1601 can include a coupling structure 1603 (individually identified as a first coupling structure 1603a on the first arm 1601a and a second coupling structure 1603b on the second arm 1601b) configured to engage the other. In the illustrated embodiment, the first coupling structure 1603a includes a spur 1605a, a notch 1609a, and a flat 1607a extending at least partially therebetween, and the second coupling structure 1603b includes a recess 1605b, a protrusion 1609b, and a flat 1607b extending therebetween. The recess 1605b can be configured to receive the spur 1605a, the notch 1609 a can be configured to receive the protrusion 1609 b, and the flats 1607a, b can be configured to be seated against one another when, e.g., the coupling structures 1603a, b engage one other. When engaged, such as shown in FIG. 16C, the coupling structures 1603a, b can be configured to resist posteriorly directed forces (including, e.g., forces applied to the coupling structures 1603a, b via the device housings 1969 because of an elastic nature of the support members 1664) but release one another in response to anteriorly-directed forces (including, e.g., forces applied to the coupling structures 1603a, b when the wearer wants to remove the wearable device 1610). For example, a posteriorly directed force applied to the coupling structures 1603a, b can cause the protrusion 1609b to pivot about the spur 1605a and move posteriorly into (e.g., further into) the recess 1605b, pressing the flats 1607a, b against each other and the spur 1605 a against a wall of the notch 1609 a, e.g., in a manner that drives the coupling structures 1603a, b, into firm (e.g., even firmer) engagement with one another. However, an anteriorly directed force applied to the coupling structures 1603a, b can cause the protrusion 1609 b to pivot about the spur 1605 a and move anterior away from (e.g., out of) the recess 1605 b, moving the flats 1607a, b away from each other and moving the spur 1605a out from the notch 1609a, e.g., in a manner that disengages the coupling structures 1603a, b.

FIG. 16D is a perspective view of the electronics housing 1636 and the support members 1664 with a cover portion of the housing 1636 and other aspects of the wearable device 1610 omitted for illustrative clarity. As best seen in FIG. 16D, in some embodiments each of the support members 1664 can be secured to the electronics housing 1636 via a buckle or anchor 1611 (individually identified as a first anchor 1611a coupled to the first support member 1664a and a second anchor 1611b coupled to the second support member 1664b). Each of the anchors 1611 can include one or more (e.g., a plurality of) posts 1613 and the housing 1636 can include or define one or more (e.g., a plurality of) of recesses 1615 configured to receive one or more of the posts 1613 on each of the anchors 1611. The recesses 1615 engaged by the posts 1613 on each anchor 1611 can be selected to increase or decrease, as needed, a length with which the corresponding support member 1664 extends beyond/outwardly from the housing 1636.

FIG. 16E is a top cross-sectional view of the wearable device 1610 positioned around a person's neck in accordance with embodiments of the present technology. When worn, the device housings 1696a, b can each be configured to contact a portion of the patient's neck overlaying/aligned with one of the patient's sternocleidomastoid muscles SCMM, respectively. The sternocleidomastoid muscles SCMM connect the wearer's head H to their sternum and serve to turn and nod the head. The sternocleidomastoid muscles SCMM also present large, generally rigid surfaces that the device housings 1696a, b can contact and act as an anatomical fiducial to, for example, register the position and/or orientation of the wearable device 1610 relative to the patient's neck. Additionally, aligning the device housings 1696a, b with the sternocleidomastoid muscles SCMM can allow the movement of the sternocleidomastoid muscles SCMM to drive corresponding movement of the device housings 1696a, b (and, by extension, movement of one or more other portions of the wearable device 1610). Each of the device housings 1696a, b is expected to move at least generally with the neck portion about which the device housings 1696a, b are seated, e.g., without or generally without slipping and/or inhibiting movement of the neck N. For example, in response to a movement of the patient's head and/or neck N, the device housings 1696a, b can follow (e.g., track) at least approximately 50%, 60%, 70%, 80%, 90%, or 100% of the movement of the sternocleidomastoid muscle SCMM and/or other neck portion along which the device housings 1696a, b are seated.

Accordingly, in some aspects of the present technology, a wearable device can include a pair of support members coupled together by connectors. The support members can be configured to be positioned along the patient's neck and track movement of the head H and/or neck N without undue restriction. For example, when a patient moves their head and/or neck, the wearable device and/or individual components coupled thereto can undergo up to 50%, 60%, 70%, 80%, 90%, or 100% of that movement. This enables consistent and easily repeatable positioning and placement of these components, which is expected to lead to increased efficacy of the therapy, lower power consumption, and higher patient compliance.

### 4. Examples

The following examples provide further embodiments of the present technology:
1. A wearable device configured to be worn about a wearer's neck, the wearable device comprising:
a housing having a first housing end portion and a second housing end portion opposite the first housing end portion;
a first support member coupled to and extending away from the first housing end portion to define a first support member end portion;
a second support member coupled to and extending away from the second housing end portion to define a second support member end portion; and a bridge portion coupled to the first support member end portion and the second support member end portion;
wherein—
   the housing is configured to contact a posterior side of the wearer's neck when the wearable device is worn,
   the first support member is configured to extend anteriorly from the housing and around one of a left or a right side of the wearer's neck when the wearable device is worn,
   the second support member is configured to extend anteriorly from the housing and around the other of the left or the right side of the wearer's neck when the wearable device is worn, and
   the bridge portion is spaced apart from the wearer's neck to define a gap between the bridge portion and an anterior side of the wearer's neck when the wearable device is worn.
2. The wearable device of example 1 wherein the housing is a first housing and wherein the wearable device further comprises:
a second housing coupled to the first support member end portion; and
a third housing coupled to the first support member end portion;
wherein the bridge portion is coupled to the first support member via the second housing and to the second support member via the third housing.
3. The wearable device of example 2 wherein the second housing is configured to contain one or more first sensors and wherein the third housing is configured to contain one or more second sensors.
4. The wearable device of example 3 wherein the one or more first sensors includes a blood oxygen sensor and wherein the one or more second sensors include an audio sensor.
5. The wearable device of any of examples 2-4 wherein the second housing is configured to be positioned over one of a left or a right sternocleidomastoid muscle in the wearer's neck and the third housing is configured to be positioned over the other of the left or the right sternocleidomastoid muscle when the wearable device is worn.
6. The wearable device of any of examples 1-5, further comprising one or more sensors, wherein individual ones of the one or more sensors are coupled to the housing, the first support member, and/or the second support member and configured to detect data associated with a sleep position, a sleep stage, and/or a breathing obstruction experienced by the wearer.
7. The wearable device of any of examples 1-6 wherein the one or more sensors include an audio sensor, a blood oxygen sensor, and an accelerometer.
8. The wearable device of any of examples 1-7 wherein the first support member and/or the second support member are configured to apply a posteriorly directed force on the bridge portion when the wearable device is worn, and wherein the bridge portion is configured to press the first support member end portion and/or the second support member end portion posteriorly against the anterior side of the wearer's neck while maintaining the gap between the bridge portion and the anterior side of the wearer's neck.
9. The wearable device of any of examples 1-8 wherein the bridge portion includes a first arm having a first coupling structure and a second arm having a second coupling structure, wherein the first coupling structure is configured to engage the second coupling structure to releasably couple the first arm to the second arm.
10. The wearable device of example 9 wherein, when engaged together, the first coupling structure and the second coupling structure are configured to resist posteriorly directed forces and disengage one another in response to anteriorly directed forces.
11. A wearable device, comprising:
a first support member positionable at least partially around a wearer's neck;
a second support member positionable at least partially around the wearer's neck superior to the first support member; and
a connector coupled between the first support member and the second support member, wherein the connector is configured to allow the first support member and the second support member to move relative to one another in response to movement of the wearer's neck,
wherein—
   a first power transmission device is carried by the first support member and positionable to transmit power to a first implantable device implanted at least proximate to an ansa cervicalis nerve of the wearer, and/or
   a second power transmission device is carried by the second support member and positionable to transmit power to a second implantable device implanted at least proximate to a hypoglossal nerve of the wearer.
12. The wearable device of example 11 wherein the connector is configured to allow the first support member and/or the second support member to rotate relative to one other in response to movement of the wearer's neck.
13. The wearable device of example 11 or example 12 wherein—
the first support member is positionable to contact a first region of the wearer's neck, the second support member is positionable to contact a second region of the wearer's neck superior to the first region, and
in response to movement of the second region of the wearer's neck relative to the first region of the wearer's neck, the connector is configured to allow the second support member to move relative to the first support member to maintain the contact between (i) the first support member and the first region and (ii) the second support member and the second region.
14. The wearable device of any of examples 11-13 wherein the connector is one of a plurality of connectors coupled between the first support member and the second support member.
15. The wearable device of any of examples 11-14 wherein the connector includes a loop of deformable material coupled between the first support member and the second support member.
16. The wearable device of any of examples 11-15 wherein the first support member and the second support member each have a C-shape that defines an anteriorly positioned opening.
17. The wearable device of any of examples 11-16 wherein the connector is a first connector and wherein the wearable device further comprises:
a third support member positioned superior to the second support member, and
a second connector coupled between the second support member and the third support member, wherein the connector is configured to allow the second support member and the third support member to move relative to one another in response to movement of the wearer's neck.
18. The wearable device of any of examples 11-17, further comprising an electronics housing coupled to one of the first support member or the second support member, wherein the electronics housing is (i) configured to be positioned on a posterior side of the neck when the wearable device is worn and/or (ii) operably coupled to the first power transmission device and/or the second power transmission device to provide power thereto for transmission to the first implantable device and/or the second implantable device.
19. The wearable device of any of examples 11-18 wherein the first support member and the second support member are positioned a distance apart from one another, and wherein the connector has a length greater than the distance between the first support member and the second support member.
20. The wearable device of any of examples 11-19 wherein the connector is configured to be held in compression between the first support member and the second support member to bias the first support member and the second support member away from one another.
21. The wearable device of any of examples 11-21, further comprising one or more sensors coupled to the first support member and/or the second support member, wherein the one or more sensors are configured to receive data associated with the wearer including a respiratory rate, a sleep state, a wake state, a heart rate, audio signals, body temperature, head orientation/position, saturated blood oxygen levels, air flow levels, thyroid movement, trachea movement, tongue movement, and/or photoplethysmography (PPG) data.
22. A power transmission device configured to be worn around a wearer's neck and wirelessly provide power to one or more implantable devices within a patient, the power transmission device comprising:
a body having a first side portion and a second side portion opposite the first side portion;
a first support member extending from the first side portion of the body and positionable around a first region of the wearer's neck, wherein the first support member includes a first end portion positioned away from the body;
a second support member extending from the second side portion of the body and positionable around a second region of the wearer's neck opposite the first side portion, wherein the second support member includes a second end portion positioned away from the body, and wherein the second end portion is spaced apart from the first end portion; and
a flexible material extending at least between the first end portion of the first support member and the second end portion of the second support member,
wherein a power transmission device is carried by the first support member or the second support member and configured to transmit power to a first implantable device implanted at least proximate to an ansa cervicalis nerve of the wearer.
23. The device of example 22 wherein the flexible material includes a covering is positioned at least partially around the first support member and/or the second support member, wherein the covering is positioned to draw the first end portion and the second end portion inwardly toward one another.
24. The device of example 22 or example 23 wherein the body includes a first connector portion and a second connector portion releasably couplable to the first connector portion.
25. The device of any of examples 22-24 wherein, when worn by the wearer, the flexible material is positioned anterior to the body.
26. The device of any of examples 22-25, further comprising: a chinrest configured support the wearer's chin and/or jaw; and a chinrest connector configured to couple the chinrest to the body.
27. The device of example 26 wherein the chinrest connector includes a plurality of joints arranged in series, wherein the plurality of joints includes—a
first joint having a first axis of rotation;
a second joint having a second axis of rotation perpendicular to the first axis of rotation; and
a third joint having a third axis of rotation perpendicular to the first axis of rotation and/or the second axis of rotation.
28. The device of example 27 wherein the second joint is in series between the first joint and the third joint.
29. The device of example 28 wherein the plurality of joints further includes a ball joint in series after the third joint.
30. The device of any of examples 26-29 wherein the chinrest connector includes a flexible shaft configured to deform in response to movement of the wearer's head.
31. The device of any of examples 26-30 wherein the chinrest connector includes a spring configured to apply an anteriorly directed force to the wearer's chin and/or jaw.
32. The device of any of examples 26-31, further comprising a second power transmission device carried by the chinrest and configured to transmit power to a second implantable device implanted at least proximate to a hypoglossal nerve of the wearer.
33. The device of any of examples 22-32, further comprising a first chin contacting portion coupled to the first support member and a second chin contacting portion coupled to the second support member, wherein, when worn by the wearer, the first chin contacting portion and/or second chin contacting portion are configured to press upwardly against an underside of the wearer's jaw.
34. The device of example 33, further comprising a second power transmission device carried by the first chin contacting portion or the second chin reset portion, wherein the second power transmission device is configured to transmit power to a second implantable device implanted at least proximate to a hypoglossal nerve of the wearer.
35. A wearable device, comprising:
a housing configured to be positioned at least partially around a posterior portion of a wearer's neck, wherein the housing portion has a first end portion and a second end portion;
a chinrest configured to be positioned at least partially inferior to the wearer's jaw, the chinrest rotatably coupled to the housing by a first joint at the first end portion and a second joint by the second end portion; and
a strap configured to extend around at least a portion of the wearer's head, the strap having a first end coupled to the chinrest proximate the first joint and a second end coupled to the chinrest proximate the second joint.
36. The wearable device of example 35 wherein, when the wearable device is worn by the wearer, the strap is configured to apply an upward force to the chinrest to engage the chinrest with the wearer's chin and/or jaw.
37. The wearable device of example 35 or example 36, further comprising a power transmission device coupled to the chinrest and configured to transmit power to an implantable device positionable within the wearer at least proximate to a hypoglossal nerve of the wearer.
38. A wearable device configured to transmit power to one or more implantable devices positioned within a wearer, the implantable device comprising:
a first support member positionable to contact a first region of a wearer's head;
a power transmission device coupled to the first support member and configured to transmit power to an implantable device positionable within the wearer at least proximate to a hypoglossal nerve of the wearer;
a second support member positionable to contact a second region of the wearer's head opposite the first region; and
a connector portion extending between the first support member and the second support member along a third region of the wearer's head between the first region and the second region, wherein the connector portion is configured to draw the first support member and the second support member toward one another.
39. The wearable device of example 38 wherein the first region of the wearer's head includes the wearer's chin and/or an underside of the wearer's jaw, and wherein the second region of the wearer's head includes a crown of the wearer's head.
40. The wearable device of example 38 or example 39 wherein the first support member includes one or more power transmission devices, wherein the one or more power transmission devices are configured to transmit power to one or more implantable devices.
41. The wearable device of any of examples 38-40 wherein the first support member, the second support member, and the connector portion together define a continuous non-planar elliptical shape.
42. The wearable device of any of examples 38-41 wherein the connector portion includes a first connector and a second connector and defines a gap between the first connector and the second connector.
43. The wearable device of example 42 wherein, when the wearable device is worn by the wearer, the gap is positionable on a posterior side of the wearer's head.
44. A wearable device operable to transmit power to one or more devices implanted within a patient, the wearable device comprising:
a first support member positionable at least partially around a wearer's neck;
a second support member positionable at least partially around the wearer's neck superior to the first support member;
a first power transmission device coupled to the first support member and positionable to transmit power to a first implantable device implanted at least proximate to an ansa cervicalis nerve of the wearer;
a second power transmission device coupled to the second support member and positionable to transmit power to a second implantable device implanted at least proximate to a hypoglossal nerve of the wearer; and
a plurality of connectors coupled between the first support member and the second support member,
wherein—
   the first support member and the second support member are positioned a distance apart from one another, and
   the connectors have a length greater than the distance between the first support member and the second support member to allow the first support member and the second support member to move relative to one another in response to movement of the wearer's neck and/or head to (i) position the first power transmission device to transmit power to the first implantable device during the movement of the wearer's neck and/or head and (ii) position the second power transmission device to transmit power to the second implantable device during the movement of the wearer's neck and/or head.
45. A wearable device operable to transmit power to one or more devices implanted within a patient, the wearable device comprising:
a primary support member positionable at least partially around a wearer's neck;
a secondary support member coupled to and extending outwardly from the primary support member in a first direction;
a tertiary support member coupled to and extending outwardly form the primary support member in a second direction, opposite the first direction; and
one or more power transmission devices, wherein individual ones of the power transmission devices are coupled to the primary, secondary, or tertiary support member and positioned to transmit power to individual ones of the one or more devices implanted within the patient.
46. The wearable device of example 45 wherein—
the secondary support member includes a first secondary end portion and a second secondary end portion opposite the first secondary end portion;
both the first secondary end portion and the second secondary end portion are coupled to the primary support member;
the tertiary support member includes a first tertiary end portion and a second tertiary end portion opposite the first tertiary end portion; and
both the first tertiary end portion and the second tertiary end portion are coupled to the primary support member.
47. The wearable device of example 45 or example 46 wherein, when the wearable device is worn by the wearer, the secondary support member includes a first loop extending superiorly outwardly from the primary support member and the tertiary support member includes a second loop extending inferiorly outwardly from the primary support member.
48. The wearable device of any one of the examples herein, wherein at least one of the power transmission devices is configured to deliver transcutaneous electrical modulation to a target tissue of the patient.
49. The wearable device of any one of the examples herein, wherein at least one of the power transmission devices is configured to deliver transcutaneous electrical modulation to an ansa cervicalis nerve of the patient.
50. The wearable device of any one of the examples herein, wherein at least one of the power transmission devices is configured to deliver transcutaneous electrical modulation to one or more muscles innervated by an ansa cervicalis nerve of the patient.
51. The wearable device of any one of the examples herein, further comprising a placement feedback system including a sensor and a feedback device configured to provide feedback to the wearer regarding the positioning of the wearable device.
52. The wearable device of example 51 wherein the sensor includes a PPG sensor, a capacitance sensor, and/or an accelerometer.
53. The wearable device of example 51 or example 52 wherein the feedback device includes a haptic feedback device.
54. The wearable device of any one of examples 51-53 wherein the placement feedback system is configured to detect longitudinal, rotational, and/or angular misplacement of the wearable device; and provide, via the feedback device, feedback to the wearer based, at least in part, on the detected misplacement.
55. The wearable device of any one of examples 51-54 wherein the placement feedback system is configured to detect longitudinal, rotational, and/or angular misplacement of the wearable device; and deactivate at least one power transmission device based, at least in part, on the detected misplacement.

### 5. Closing Remarks

It will be appreciated that specific embodiments of the disclosed technology have been described herein for purposes of illustration, but that various modifications may be made without deviating from the technology. For example, the signal delivery device can be leadless or can include a lead with one or more of the electrodes of the signal delivery device carried by the lead. Certain aspects of the technology described in the context of particular embodiments may be combined or eliminated in other embodiments. For example, two signal delivery devices can be implanted to bilaterally target the patient's tissues (e.g., left and right ansa cervicalis nerves) and/or to target different tissues on left and right sides of the patient (e.g., a left ansa cervicalis nerve and a right infrahyoid strap muscle of the patient). Further, while advantages associated with certain embodiments of the disclosed technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Several modulation targets and/or implantation techniques are described and/or illustrated with reference to at least FIGS. 1A, 1B, 2B, and 2C. For the purpose of illustrative clarity, these modulation targets and/or implantation techniques are shown with reference to a left or right side of the patient P's anatomy, for example, a first or left ansa cervicalis nerve of the patient P and/or a first or left one of the infrahyoid strap muscles. It will be appreciated, however, that at least some or all of the modulation targets and/or implantation techniques described and/or illustrated with reference to at least FIGS. 1A, 1B, 2B, and 2C are equally suitable for application to the other side of the patient's anatomy, for example, the second or right ansa cervicalis nerve of the patient P and/or the second or right one of the infrahyoid strap muscles. Additionally, at least some of the modulation targets and/or implantation techniques can be used for bilateral signal delivery, for example, to apply a first modulation signal to a first modulation target on a first side of the patient P at a first time, and to apply a second modulation signal to a second modulation target on a side of the patient P at the same or different time. The second modulation signal can be the same or different than the first modulation signal. In some embodiments, the first and second modulation targets can be corresponding left and right portions of the patient's anatomy, such as first and second portions of the left and right ansa cervicalis nerves. In other embodiments, the first and second modulation targets can be different, such as the left ansa cervicalis nerve and an infrahyoid strap muscle on a right side of the patient. Additional details regarding placing signal delivery devices to deliver modulation signals to the hypoglossal nerve are described in U.S. Pat. App. No. Ser. No. 18/393,537, filed Dec. 21, 2023, the entirety of which is hereby incorporated by reference herein. Additional details regarding placing signal delivery devices to deliver modulation signals to the ansa cervicalis are described in U.S. App. No. Ser. No. 18/607,289, filed Mar. 15, 2024, the entirety of which is hereby incorporated by reference herein.

As used herein, the phrase “and/or,” as in “A” and/or “B” refers to A alone, B alone and both A and B. Unless otherwise stated, the terms “generally,” “about,” and “approximately” refer to values within 10% of a stated value. For example, the use of the term “about 100” refers to a range of 90 to 110, inclusive. In instances in which the context requires otherwise and/or relative terminology is used in reference to something that does not include a numerical value, the terms are given their ordinary meaning to one skilled in the art.

To the extent any materials incorporated herein by reference conflict with the present disclosure, the present disclosure controls.

I/We claim:

1. A wearable device configured to be worn about a wearer's neck, the wearable device comprising:
a housing having a first housing end portion and a second housing end portion opposite the first housing end portion;
a first support member coupled to and extending away from the first housing end portion to define a first support member end portion;
a second support member coupled to and extending away from the second housing end portion to define a second support member end portion; and
a bridge portion coupled to the first support member end portion and the second support member end portion;
wherein—
the housing is configured to contact a posterior side of the wearer's neck when the wearable device is worn,
the first support member is configured to extend anteriorly from the housing and around one of a left or a right side of the wearer's neck when the wearable device is worn,
the second support member is configured to extend anteriorly from the housing and around the other of the left or the right side of the wearer's neck when the wearable device is worn, and
the bridge portion is spaced apart from the wearer's neck to define a gap between the bridge portion and an anterior side of the wearer's neck when the wearable device is worn.

2. The wearable device of claim 1 wherein the housing is a first housing and wherein the wearable device further comprises:
a second housing coupled to the first support member end portion; and
a third housing coupled to the first support member end portion;
wherein the bridge portion is coupled to the first support member via the second housing and to the second support member via the third housing.

3. The wearable device of claim 2 wherein the second housing is configured to contain one or more first sensors and wherein the third housing is configured to contain one or more second sensors.

4. The wearable device of claim 3 wherein the one or more first sensors includes a blood oxygen sensor and wherein the one or more second sensors include an audio sensor.

5. The wearable device of claim 2 wherein the second housing is configured to be positioned over one of a left or a right sternocleidomastoid muscle in the wearer's neck and the third housing is configured to be positioned over the other of the left or the right sternocleidomastoid muscle when the wearable device is worn.

6. The wearable device of claim 1, further comprising one or more sensors, wherein individual ones of the one or more sensors are coupled to the housing, the first support member, and/or the second support member and configured to detect data associated with a sleep position, a sleep stage, and/or a breathing obstruction experienced by the wearer.

7. The wearable device of claim 1 wherein the one or more sensors include an audio sensor, a blood oxygen sensor, and an accelerometer.

8. The wearable device of claim 1 wherein the first support member and/or the second support member are configured to apply a posteriorly directed force on the bridge portion when the wearable device is worn, and wherein the bridge portion is configured to press the first support member end portion and/or the second support member end portion posteriorly against the anterior side of the wearer's neck while maintaining the gap between the bridge portion and the anterior side of the wearer's neck.

9. The wearable device of claim 1 wherein the bridge portion includes a first arm having a first coupling structure and a second arm having a second coupling structure, wherein the first coupling structure is configured to engage the second coupling structure to releasably couple the first arm to the second arm.

10. The wearable device of claim 9 wherein, when engaged together, the first coupling structure and the second coupling structure are configured to resist posteriorly directed forces and disengage one another in response to anteriorly directed forces.

11. A wearable device, comprising:
a first support member positionable at least partially around a wearer's neck;
a second support member positionable at least partially around the wearer's neck superior to the first support member; and
a connector coupled between the first support member and the second support member, wherein the connector is configured to allow the first support member and the second support member to move relative to one another in response to movement of the wearer's neck, wherein—
a first power transmission device is carried by the first support member and positionable to transmit power to a first implantable device implanted at least proximate to an ansa cervicalis nerve of the wearer, and/or a second power transmission device is carried by the second support member and positionable to transmit power to a second implantable device implanted at least proximate to a hypoglossal nerve of the wearer.

12. The wearable device of claim 11 wherein the connector is configured to allow the first support member and/or the second support member to rotate relative to one other in response to movement of the wearer's neck.

13. The wearable device of claim 11 wherein—
the first support member is positionable to contact a first region of the wearer's neck,
the second support member is positionable to contact a second region of the wearer's neck superior to the first region, and
in response to movement of the second region of the wearer's neck relative to the first region of the wearer's neck, the connector is configured to allow the second support member to move relative to the first support member to maintain the contact between (i) the first support member and the first region and (ii) the second support member and the second region.

14. The wearable device of claim 11 wherein the connector is one of a plurality of connectors coupled between the first support member and the second support member.

15. The wearable device of claim 11 wherein the connector includes a loop of deformable material coupled between the first support member and the second support member.

16. The wearable device of claim 11 wherein the first support member and the second support member each have a C-shape that defines an anteriorly positioned opening.

17. The wearable device of claim 11 wherein the connector is a first connector and wherein the wearable device further comprises:
a third support member positioned superior to the second support member, and
a second connector coupled between the second support member and the third support member, wherein the connector is configured to allow the second support member and the third support member to move relative to one another in response to movement of the wearer's neck.

18. The wearable device of claim 11, further comprising an electronics housing coupled to one of the first support member or the second support member, wherein the electronics housing is (i) configured to be positioned on a posterior side of the neck when the wearable device is worn and/or (ii) operably coupled to the first power transmission device and/or the second power transmission device to provide power thereto for transmission to the first implantable device and/or the second implantable device.

19. The wearable device of claim 11 wherein the first support member and the second support member are positioned a distance apart from one another, and wherein the connector has a length greater than the distance between the first support member and the second support member.

20. The wearable device of claim 11 wherein the connector is configured to be held in compression between the first support member and the second support member to bias the first support member and the second support member away from one another.

21. The wearable device of claim 11, further comprising one or more sensors coupled to the first support member and/or the second support member, wherein the one or more sensors are configured to receive data associated with the wearer including a respiratory rate, a sleep state, a wake state, a heart rate, audio signals, body temperature, head orientation/position, saturated blood oxygen levels, air flow levels, thyroid movement, trachea movement, tongue movement, and/or photoplethysmography (PPG) data.

22. A power transmission device configured to be worn around a wearer's neck and wirelessly provide power to one or more implantable devices within a patient, the power transmission device comprising:
a body having a first side portion and a second side portion opposite the first side portion;
a first support member extending from the first side portion of the body and positionable around a first region of the wearer's neck, wherein the first support member includes a first end portion positioned away from the body;
a second support member extending from the second side portion of the body and positionable around a second region of the wearer's neck opposite the first side portion, wherein the second support member includes a second end portion positioned away from the body, and wherein the second end portion is spaced apart from the first end portion; and
a flexible material extending at least between the first end portion of the first support member and the second end portion of the second support member,
wherein a power transmission device is carried by the first support member or the second support member and configured to transmit power to a first implantable device implanted at least proximate to an ansa cervicalis nerve of the wearer.

23. The device of claim 22 wherein the flexible material includes a covering is positioned at least partially around the first support member and/or the second support member, wherein the covering is positioned to draw the first end portion and the second end portion inwardly toward one another.

24. The device of claim 22 wherein the body includes a first connector portion and a second connector portion releasably couplable to the first connector portion.

25. The device of claim 22 wherein, when worn by the wearer, the flexible material is positioned anterior to the body.

26. The device of claim 22, further comprising:
a chinrest configured support the wearer's chin and/or jaw; and
a chinrest connector configured to couple the chinrest to the body.

27. The device of claim 26 wherein the chinrest connector includes a plurality of joints arranged in series, wherein the plurality of joints includes—
a first joint having a first axis of rotation;
a second joint having a second axis of rotation perpendicular to the first axis of rotation; and
a third joint having a third axis of rotation perpendicular to the first axis of rotation and/or the second axis of rotation.

28. The device of claim 27 wherein the second joint is in series between the first joint and the third joint.

29. The device of claim 28 wherein the plurality of joints further includes a ball joint in series after the third joint.

30. The device of claim 26 wherein the chinrest connector includes a flexible shaft configured to deform in response to movement of the wearer's head.

31. The device of claim 26 wherein the chinrest connector includes a spring configured to apply an anteriorly directed force to the wearer's chin and/or jaw.

32. The device of claim 26, further comprising a second power transmission device carried by the chinrest and configured to transmit power to a second implantable device implanted at least proximate to a hypoglossal nerve of the wearer.

33. The device of claim 22, further comprising a first chin contacting portion coupled to the first support member and a second chin contacting portion coupled to the second support member, wherein, when worn by the wearer, the first chin contacting portion and/or second chin contacting portion are configured to press upwardly against an underside of the wearer's jaw.

34. The device of claim 33, further comprising a second power transmission device carried by the first chin contacting portion or the second chin reset portion, wherein the second power transmission device is configured to transmit power to a second implantable device implanted at least proximate to a hypoglossal nerve of the wearer.

35. A wearable device, comprising:
a housing configured to be positioned at least partially around a posterior portion of a wearer's neck, wherein the housing portion has a first end portion and a second end portion;
a chinrest configured to be positioned at least partially inferior to the wearer's jaw, the chinrest rotatably coupled to the housing by a first joint at the first end portion and a second joint by the second end portion; and
a strap configured to extend around at least a portion of the wearer's head, the strap having a first end coupled to the chinrest proximate the first joint and a second end coupled to the chinrest proximate the second joint.

36. The wearable device of claim 35 wherein, when the wearable device is worn by the wearer, the strap is configured to apply an upward force to the chinrest to engage the chinrest with the wearer's chin and/or jaw.

37. The wearable device of claim 35, further comprising a power transmission device coupled to the chinrest and configured to transmit power to an implantable device positionable within the wearer at least proximate to a hypoglossal nerve of the wearer.

38. A wearable device configured to transmit power to one or more implantable devices positioned within a wearer, the implantable device comprising:
a first support member positionable to contact a first region of a wearer's head;
a power transmission device coupled to the first support member and configured to transmit power to an implantable device positionable within the wearer at least proximate to a hypoglossal nerve of the wearer;
a second support member positionable to contact a second region of the wearer's head opposite the first region; and
a connector portion extending between the first support member and the second support member along a third region of the wearer's head between the first region and the second region, wherein the connector portion is configured to draw the first support member and the second support member toward one another.

39. The wearable device of claim 38 wherein the first region of the wearer's head includes the wearer's chin and/or an underside of the wearer's jaw, and wherein the second region of the wearer's head includes a crown of the wearer's head.

40. The wearable device of claim 38 wherein the first support member includes one or more power transmission devices, wherein the one or more power transmission devices are configured to transmit power to one or more implantable devices.

41. The wearable device of claim 38 wherein the first support member, the second support member, and the connector portion together define a continuous non-planar elliptical shape.

42. The wearable device of claim 38 wherein the connector portion includes a first connector and a second connector and defines a gap between the first connector and the second connector.

43. The wearable device of claim 42 wherein, when the wearable device is worn by the wearer, the gap is positionable on a posterior side of the wearer's head.

44. A wearable device operable to transmit power to one or more devices implanted within a patient, the wearable device comprising:
a first support member positionable at least partially around a wearer's neck;
a second support member positionable at least partially around the wearer's neck superior to the first support member;
a first power transmission device coupled to the first support member and positionable to transmit power to a first implantable device implanted at least proximate to an ansa cervicalis nerve of the wearer;
a second power transmission device coupled to the second support member and positionable to transmit power to a second implantable device implanted at least proximate to a hypoglossal nerve of the wearer; and
a plurality of connectors coupled between the first support member and the second support member,
wherein—
the first support member and the second support member are positioned a distance apart from one another, and
the connectors have a length greater than the distance between the first support member and the second support member to allow the first support member and the second support member to move relative to one another in response to movement of the wearer's neck and/or head to (i) position the first power transmission device to transmit power to the first implantable device during the movement of the wearer's neck and/or head and (ii) position the second power transmission device to transmit power to the second implantable device during the movement of the wearer's neck and/or head.

45. A wearable device operable to transmit power to one or more devices implanted within a patient, the wearable device comprising:
a primary support member positionable at least partially around a wearer's neck;
a secondary support member coupled to and extending outwardly from the primary support member in a first direction;
a tertiary support member coupled to and extending outwardly form the primary support member in a second direction, opposite the first direction; and
one or more power transmission devices, wherein individual ones of the power transmission devices are coupled to the primary, secondary, or tertiary support member and positioned to transmit power to individual ones of the one or more devices implanted within the patient.

46. The wearable device of claim 45 wherein—
the secondary support member includes a first secondary end portion and a second secondary end portion opposite the first secondary end portion;
both the first secondary end portion and the second secondary end portion are coupled to the primary support member;
the tertiary support member includes a first tertiary end portion and a second tertiary end portion opposite the first tertiary end portion; and
both the first tertiary end portion and the second tertiary end portion are coupled to the primary support member.

47. The wearable device of claim 45 wherein, when the wearable device is worn by the wearer, the secondary support member includes a first loop extending superiorly outwardly from the primary support member and the tertiary support member includes a second loop extending inferiorly outwardly from the primary support member.
